(19) United States
(12) Patent Application Publication
(10) Pub. No.: US 2020/0291449 A1
(43) Pub. Date: Sep. 17, 2020

(54) METHODS AND COMPOSITIONS FOR DETECTING ANTI-DRUG ANTIBODIES

(71) Applicant: ALNYLAM PHARMACEUTICALS, INC., Cambridge, MA (US)

(72) Inventors: Kallanthottathil G. Rajeev, Wayland, MA (US); Jayaprakash Nair, Cambridge, MA (US); Muthiah Manoharan, Cambridge, MA (US); Renta Hutabarat, Cambridge, MA (US); Minggeng Gao, Cambridge, MA (US)

(21) Appl. No.: 16/804,777

(22) Filed: Feb. 28, 2020

Related U.S. Application Data
(63) Continuation of application No. 15/305,830, filed on Oct. 21, 2016, now abandoned.
(60) Provisional application No. 61/983,791, filed on Apr. 24, 2014.

(51) Int. Cl. C12Q 1/6804; C12Q 1/68; G01N 33/53; C07K 16/44

(52) U.S. Cl. CPC C12Q 1/6804; C12Q 1/68; C07K 2317/33; C07K 16/44; C07K 2317/22; G01N 33/5308

(57) ABSTRACT

Assays, methods, reagents and kits for evaluating the level of an antibody against a nucleic acid molecule, e.g., a double-stranded oligonucleotide or RNA molecule (e.g., dsRNA), are disclosed herein.

### CROSS REFERENCE TO RELATED APPLICATIONS

This application is a continuation of U.S. Application Ser. No. 15/305,830, filed Oct. 21, 2016, which is a U.S. National Stage Application under 35 U.S.C. § 371 of International Application No. PCT/US2015/027341, filed Apr. 23, 2015, which claims the benefit of priority to U.S. Provisional Application Ser. No. 61/983,791, filed Apr. 24, 2014. The contents of the aforesaid applications are hereby incorporated by reference in their entirety.

### BACKGROUND OF THE INVENTION

Biopharmaceutical products (e.g., proteins, carbohydrates and nucleic acids) can elicit an immune response in a patient receiving a treatment. The immunogenic potential of a biopharmaceutical product can be associated with various factors, including, but not limited to, product intrinsic factors, product extrinsic factors and patient-specific factors. Exemplary product intrinsic factors include species-specific epitopes (such as, degree of foreignness), glycosylation status, extent of aggregation or denaturation, impurities, and formulation. Examples of product extrinsic factors include route of administration, acute or chronic dosing, pharmacokinetics, and existence of endogenous equivalents. Examples of patient-specific factors include autoimmune disease, immunosuppression, and replacement therapy. The induction of anti-drug antibodies (ADAs) can result in adverse clinical responses such as hypersensitivity and autoimmunity, as well as altered pharmacokinetics (e.g., drug neutralization, abnormal biodistribution, and enhanced drug clearance rates). These clinical responses can alter the efficacy of the treatment. Therefore, immune responses caused by biopharmaceuticals can be an important safety and efficacy concern for regulatory agencies, drug manufacturers, clinicians, and patients.

Thus, the need exists for developing novel assays, methods, and compounds for detecting anti-drug antibodies for biopharmaceutical products, such as nucleic acid products, e.g., RNA molecules.

### SUMMARY OF THE INVENTION

Disclosed herein are assays, methods, compositions and kits for evaluating, e.g., detecting the level of, an antibody against a nucleic acid molecule (e.g., an oligonucleotide molecule (e.g., a single stranded- or a double-stranded oligonucleotide), or an RNA molecule, e.g., a single stranded- or a double-stranded RNA (dsRNA)). In one embodiment, the nucleic acid molecule is immobilized, e.g., directly or indirectly, to a solid support. For example, Applicants have discovered that immobilization, e.g., covalent immobilization, of a nucleic acid (e.g., double-stranded oligonucleotide or RNA molecule, e.g., dsRNA) molecule to a solid support provides a stable, qualitative and quantifiable display of a substantially non-denatured nucleic acid molecule. In another embodiment, the nucleic acid molecule (e.g., double-stranded oligonucleotide or RNA molecule, e.g., dsRNA) is immobilized to a solid support via a binding agent, e.g., an antibody molecule. Contacting of the immobilized nucleic acid molecule (e.g., double-stranded oligonucleotide or RNA molecule, e.g., dsRNA) with a sample (e.g., a plasma sample, a serum sample or a whole blood sample from a subject) can be effected under conditions that allow binding of the antibody against the nucleic acid molecule (“anti-nucleic acid molecule antibody), if present in the sample, to the immobilized double stranded oligonucleotides or nucleic acid molecule, thereby forming a complex between the nucleic acid molecule antibody and the immobilized nucleic acid molecule. Optionally, a detection agent that specifically binds to the complex of the anti- nucleic acid molecule antibody and the immobilized nucleic acid molecule can be added. In the aforesaid embodiments, the contacting step is effected in a solid support, e.g., using an enzyme-linked immunosorbent assay (ELISA). Alternatively described herein are embodiments where the contacting step is effected in solution, e.g., using a radioimmunoassay (RIA). Additionally disclosed herein are binding agents that can be used in the detection, calibration and/or quantification of the antibodies, the nucleic acid molecules (e.g., double-stranded oligonucleotide or RNA molecule, e.g., dsRNA). In one embodiment, the binding agent is an antibody molecule that binds to the nucleic acid molecule (e.g., double-stranded oligonucleotide or RNA molecule, e.g., dsRNA). The assays, methods, binding agents, compositions and kits described herein can be used, for example, to detect an antibody response to a nucleic acid molecule, e.g., an anti-drug antibody (ADA) response, in a subject.

Accordingly, in one aspect, the invention features an assay, or a method, for evaluating, e.g., detecting, an antibody against a nucleic acid molecule (e.g., an oligonucleotide molecule (e.g., a single stranded- or a double-stranded oligonucleotide), or an RNA molecule, e.g., a single stranded- or a double-stranded RNA (dsRNA)). The method includes:

(a) providing a nucleic acid molecule (e.g., double-stranded oligonucleotide or RNA molecule, e.g., dsRNA) immobilized, e.g., directly or indirectly, to a solid support;

(b) contacting said immobilized nucleic acid molecule with a sample (e.g., a sample acquired from a subject) under conditions that allow binding of the antibody against the nucleic acid molecule (“anti-nucleic acid molecule antibody”), if present in the sample, to the immobilized nucleic acid molecule (e.g., double-stranded oligonucleotide or RNA molecule, e.g., dsRNA), thereby forming a complex of the anti-nucleic acid molecule (e.g., double-stranded oligonucleotide or RNA molecule, e.g., dsRNA) antibody and the immobilized nucleic acid molecule; and

(c) (optionally) providing a detection agent that specifically binds to the complex of the anti- nucleic acid molecule antibody and the immobilized nucleic acid molecule (e.g., double-stranded oligonucleotide or RNA molecule, e.g., dsRNA) under conditions where binding to the complex, if present, occurs, thereby allowing detection of the bound anti-nucleic acid molecule antibody.

In another aspect, the invention features a method for evaluating (e.g., detecting, or monitoring, the level of) an anti-drug antibody (ADA) to a nucleic acid molecule (e.g., an oligonucleotide molecule (e.g., a single stranded- or a double-stranded oligonucleotide), or an RNA molecule, e.g., a single stranded- or a double-stranded RNA (dsRNA)), in a subject. The method includes:

(a) providing a sample (e.g., a sample acquired from a subject (e.g., a subject who has undergone, is undergoing or will receive a therapy that comprises the double stranded oligonucleotides or nucleic acid molecule));

(b) contacting said sample with an immobilized form of the nucleic acid molecule (e.g., double-stranded oligonucleotide or RNA molecule, e.g., dsRNA) under conditions that allow binding of the ADA, if present in the sample, to the immobilized nucleic acid molecule, thereby forming a complex of the ADA and the immobilized nucleic acid molecule (e.g., double-stranded oligonucleotide or RNA molecule, e.g., dsRNA); and

(c) (optionally) detecting the complex of the ADA and the immobilized nucleic acid molecule (e.g., double-stranded oligonucleotide or RNA molecule, e.g., dsRNA) under conditions where binding to the complex is indicative of the presence of the ADA, thereby allowing evaluation (e.g., detection or monitoring) of the ADA (e.g., level of ADA) in the subject. In certain embodiments, the nucleic acid molecule (e.g., double-stranded oligonucleotide or RNA molecule, e.g., dsRNA) is immobilized, e.g., directly or indirectly, to a solid support.

In the aforesaid methods, the contacting step is effected in a solid support, e.g., using an enzyme-linked immunosorbent assay (ELISA). An exemplary assay format is depicted in FIG. 1.

In another aspect, the invention features a kit for evaluating, e.g., detecting, an antibody against a nucleic acid molecule (e.g., an oligonucleotide molecule (e.g., a single stranded- or a double-stranded oligonucleotide), or an RNA molecule, e.g., a single stranded- or a double-stranded RNA (dsRNA)) (e.g., an anti-drug antibody (ADA)), in a sample. The kit includes:

(a) a nucleic acid molecule (e.g., double-stranded oligonucleotide or RNA molecule, e.g., dsRNA) immobilized to a solid support;

(b) (optionally) a detection agent that specifically binds to a complex of the antibody and the immobilized nucleic acid molecule;

(c) instructions for contacting said immobilized double stranded oligonucleotides or nucleic acid molecule with the sample under conditions that allow binding of the antibody, if present in the sample, to the immobilized nucleic acid molecule (e.g., double-stranded oligonucleotide or RNA molecule, e.g., dsRNA), and (optionally) instructions for detecting the complex of the antibody and the immobilized nucleic acid molecule (e.g., double-stranded oligonucleotide or RNA molecule, e.g., dsRNA).

Additionally disclosed herein are methods of providing an immobilized nucleic acid molecule (e.g., an oligonucleotide molecule (e.g., a single stranded- or a double-stranded oligonucleotide), or an RNA molecule, e.g., a single stranded- or a double-stranded RNA (dsRNA)) to a solid support. In one embodiment, a method of providing a covalently immobilized nucleic acid molecule to a solid support is disclosed. The method includes:

a) providing the nucleic acid molecule (e.g., double-stranded oligonucleotide or dsRNA);

b) modifying, e.g., phosphorylating, an end, e.g., 5′-end, of a sense or an antisense strand, or both, of the nucleic molecule;

c) immobilizing the modified, e.g., phosphorylated, end of the nucleic molecule to the solid support via a reactive group present on the solid support. In one embodiment, the reactive group is chosen from an amine (e.g., secondary amino) group or a sulfhydryl group.

In one embodiment, the immobilization of the nucleic acid molecule (e.g., double-stranded oligonucleotide or RNA molecule, e.g., dsRNA) to the solid support provides one or more of a stable nucleic acid molecule, a qualitative display of the nucleic acid molecule, a quantitative display of the nucleic acid molecule, a substantially non-denatured nucleic acid molecule, or a nucleic acid molecule conformation that exposes one or more epitopes.

In one embodiment, the phosphate group of the RNA or nucleic acid molecule forms a covalent bond with the reactive group. For example, the phosphate group of the RNA molecule can form a phosphoramidate bond with the secondary amino group present on the solid support.

In one embodiment, the solid support is a polystyrene surface. The polystyrene surface can be grafted with one or more secondary amino groups.

Immobilized double stranded oligonucleotides or nucleic acid molecules (e.g., a single stranded- or a double-stranded oligonucleotide), or an RNA molecule, e.g., a single stranded- or a double-stranded RNA (dsRNA) made as described herein, e.g., made by the methods described herein are also within the scope of the invention.

Other aspects feature a binding agent that can be used in the detection, calibration and/or quantification of the antibodies or the nucleic acid molecules (e.g., a single stranded- or a double-stranded oligonucleotide), or an RNA molecule, e.g., a single stranded- or a double-stranded RNA (dsRNA). In one embodiment, the binding agent is an antibody molecule that binds to the nucleic acid molecule (e.g., double-stranded oligonucleotide or RNA molecule, e.g., dsRNA). For example, the binding agent can be an antibody molecule that binds in a sequence-specific manner to an RNA molecule, e.g., a dsRNA. In other embodiments, the binding agent is an antibody molecule that binds to a modified RNA molecule, e.g., a fluoro group (e.g., a fluoro group in the 2′-position of a ribonucleotide) of the RNA molecule; or a ligand in a conjugate of the RNA molecule, e.g., a ligand that includes one or more N-acetylgalactosamine (GalNAc) ligands. In one embodiment, the binding agent, e.g., the antibody molecule, is used as a control, e.g., a positive control, in the methods and assays described herein.

In yet another aspect, the invention features a method for evaluating, e.g., detecting, an antibody against a nucleic acid molecule (e.g., an oligonucleotide molecule (e.g., a double-stranded oligonucleotide), or an RNA molecule, e.g., a double-stranded RNA (dsRNA)), e.g., an anti-drug antibody (ADA). The method includes:

(a) providing the double stranded oligonucleotides or nucleic acid molecule;

(b) providing a pre-determined amount of a binding agent, e.g., an antibody molecule, that binds to the nucleic acid molecule (e.g., the double-stranded oligonucleotide or RNA molecule, e.g., dsRNA) (e.g., an antibody molecule as described herein), wherein either the nucleic acid molecule or the binding agent, or both are detectably labeled (e.g., radioactively- or fluorescently-labeled),

(c) combining, e.g., in solution, the nucleic acid molecule and the binding agent in the presence or the absence of a sample (e.g., a sample acquired from a subject) under conditions that allow binding of either the binding agent or the antibody, if present in the sample, to the nucleic acid molecule to occur,

thereby evaluating, e.g., detecting, the antibody against the nucleic molecule, e.g., ADA, in solution.

In certain embodiments, the method further comprises determining the amount of a complex between the nucleic acid molecule (e.g., the double-stranded oligonucleotide or RNA molecule, e.g., dsRNA) and the binding agent, wherein a decrease in said complex is indicative of the level (e.g., presence or amount) of the antibody against the nucleic acid molecule (e.g., double-stranded oligonucleotide or RNA molecule, e.g., dsRNA) in the sample. In certain embodiments, the amount of the complex between the nucleic acid molecule (e.g., double-stranded oligonucleotide or RNA molecule, e.g., dsRNA) and the binding agent is determined as an inverse of the amount of the free nucleic acid molecule (e.g., double-stranded oligonucleotide or RNA molecule, e.g., dsRNA) or the binding agent detected. For example, if the binding agent is detectably-labeled, the amount of free binding agent is indicative of the amount of the antibody to the nucleic acid molecule (e.g., double-stranded oligonucleotide or RNA molecule, e.g., dsRNA) present in the sample.

In the aforesaid embodiment, the combining step is effected in solution, e.g., using a radioimmunoassay (RIA). Other alternative methods and assays for determining a binding interaction can be used, for example, Surface Plasmon Resonance (e.g., BIAcore).

In certain embodiments, the binding agent is an antibody molecule that that binds in a sequence-specific manner to an RNA molecule, e.g., a dsRNA. In other embodiments, the binding agent binds to a modified RNA molecule, e.g., a fluoro group (e.g., a fluoro group in the 2′-position of a ribonucleotide) of the RNA molecule; or a ligand in a conjugate of the RNA molecule, e.g., a ligand that includes one or more N-acetylgalactosamine (GalNAc) ligands. In certain embodiments, the binding agent is detectably-labeled (e.g., radioactively- or fluorescently-labeled).

Other features and embodiments of the methods, assays, kits and binding agents of invention include one or more of the following:

### Nucleic Acid, e.g., RNA, Molecules

In some embodiments, the nucleic acid (e.g., RNA) molecule is chosen from: a double stranded oligonucleotide , a double stranded RNA (dsRNA) molecule, a single-stranded oligonucleotide, a single-stranded RNA (e.g., RNAi) molecule, a microRNA (miRNA), an antisense RNA, a short hairpin RNA (shRNA), iRNA or an mRNA. In certain embodiments, the nucleic acid molecule (e.g., an oligonucleotide molecule (e.g., a double-stranded oligonucleotide), or an RNA molecule, e.g., a double-stranded RNA (dsRNA)) includes a conjugate of an RNA molecule and a ligand, e.g., a carbohydrate ligand (e.g., a ligand that includes one or more N-acetylgalactosamine (GalNAc) ligands). Each of these double stranded oligonucleotides or nucleic acid molecules is described in more detail below.

In some embodiments, the nucleic acid molecule is chosen from: a double stranded oligonucleotide, a double stranded RNA (dsRNA) molecule, a single-stranded RNAi molecule, a microRNA (miRNA), an antisense RNA, a short hairpin RNA (shRNA), iRNA, an antagomir, an mRNA, a decoy RNA, a DNA, a plasmids or an aptamer. In one embodiment, the nucleic acid molecule is an RNA molecule, e.g., an RNA molecule as described herein (e.g., an RNA molecule capable of mediating RNA interference or an iRNA). In one embodiment, the RNA molecule is double-stranded (e.g., a dsRNA). In embodiments, the RNA molecule comprises a sense and an antisense strand. For example, the RNA molecule is a dsRNA that forms a duplex structure between 15 and 30 base pairs in length. In one embodiment, the region of complementarity between the strands is at least 17 nucleotides in length (e.g., between 19 and 25, e.g., between 19 to 21, nucleotides in length). In some embodiments, each strand of the nucleic acid (e.g., RNA) molecule is no more than 30 nucleotides in length.

In other embodiments, the nucleic acid (e.g., RNA) molecules described herein encompass a double stranded oligonucleotide or a dsRNA having an RNA strand (the antisense strand) having a region, e.g., a region that is 30 nucleotides or less, generally 19-24 nucleotides in length, that is substantially complementary to at least part of a target mRNA.

In other embodiments, the nucleic acid (e.g., RNA) molecule is a single-stranded molecule, e.g., comprises an antisense strand.

In some embodiments, the nucleic acid molecule (e.g., double-stranded oligonucleotide or RNA molecule, e.g., dsRNA) is 19-21 nucleotides in length. In some embodiments, the iRNA is 19-21 nucleotides in length and is in a lipid formulation, e.g. a lipid nanoparticle (LNP) formulation (e.g., an LNP11 formulation).

In other embodiments, the nucleic acid molecule (e.g., double-stranded oligonucleotide or RNA molecule, e.g., dsRNA) is 21-23 nucleotides in length.

In some embodiments, the nucleic acid molecule (e.g., double-stranded oligonucleotide or RNA molecule, e.g., dsRNA) is from about 15 to about 25 nucleotides in length, and in other embodiments the nucleic acid molecule (e.g., double-stranded oligonucleotide or RNA molecule, e.g., dsRNA) is from about 25 to about 30 nucleotides in length. The nucleic acid molecule can inhibit the expression of a target gene by at least 10%, at least 20%, at least 25%, at least 30%, at least 35% or at least 40% or more, such as when assayed by a method as described herein.

In other embodiments, the nucleic acid molecule (e.g., double-stranded oligonucleotide or RNA molecule, e.g., dsRNA) comprises at least one modified nucleotide. The modified nucleotide can be chosen from one or more of: a 2′-O-methyl modified nucleotide, a nucleotide comprising a 5′-phosphorothioate group, and a terminal nucleotide linked to a cholesteryl derivative or dodecanoic acid bisdecylamide group; or a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

In other embodiments, least one strand of the nucleic acid molecule (e.g., double-stranded oligonucleotide or RNA molecule, e.g., dsRNA) comprises a 3′ overhang of at least 2 nucleotides. In other embodiments, one end of the double-stranded molecule is blunt-ended.

In embodiments, the nucleic acid molecule (e.g., double-stranded oligonucleotide or RNA molecule, e.g., dsRNA) has a sequence having an identity of at least 70 percent (e.g., 80%, 90%, 95% or higher) to a target mRNA. In one embodiment, the nucleic acid molecule (e.g., double-stranded oligonucleotide or RNA molecule, e.g., dsRNA) has a sequence complementary (e.g., is fully complementary or substantially complementary) to a target mRNA.

In one embodiment, the nucleic acid molecule (e.g., double-stranded oligonucleotide or RNA molecule, e.g., dsRNA), is in the form of a conjugate. In certain embodiments, the nucleic acid molecule (e.g., double-stranded oligonucleotide or RNA molecule, e.g., dsRNA) is coupled to (directly or via a linker) to one or more ligands or moieties, which may confer a functionality, e.g., by altering (e.g., enhancing) one or more of the activity, cellular distribution or cellular uptake of the nucleic acid molecule. The conjugate can be attached to a ligand or moiety at any suitable location in the nucleic acid molecule, e.g., at the 3′-end, the 5′-end, or both, of the sense and/or the antisense strand. In one embodiment, the ligand or moiety is attached at the 3′-end of the sense strand. In one embodiment, the ligand or moiety is attached at the 3′-end of the sense strand of a blunt-ended oligonucleotide or dsRNA molecule.

In some embodiments, the ligand includes a carbohydrate or a lipid. In one embodiment, the ligand includes one or more N-acetylgalactosamine (GalNAc) ligands. In some embodiments, the GalNAc conjugate serves as a ligand that targets the nucleic acid molecule (e.g., double-stranded oligonucleotide or RNA molecule, e.g., dsRNA) to a particular cell. In some embodiments, the GalNAc conjugate targets the RNA molecule (e.g., iRNA) to a liver cell, e.g., by serving as a ligand for the asialoglycoprotein receptor of the liver cells (e.g., hepatocytes).

In some embodiments, the carbohydrate conjugate comprises one or more GalNAc derivatives. For example, the ligand has a single GalNAc ligand, two GalNAc ligands, or three GalNAc ligands (e.g., a triantennary GalNAc ligand (GalNAc₃). The GalNAc derivatives may be attached via a linker, e.g., a bivalent or trivalent branched linker. In some embodiments the GalNAc conjugate is conjugated to the 3′ end of the sense strand. In some embodiments, the GalNAc conjugate is conjugated to the iRNA agent (e.g., to the 3′ end of the sense strand) via a linker, e.g., a linker as described herein.

In some embodiments, the GalNAc conjugate include the following:

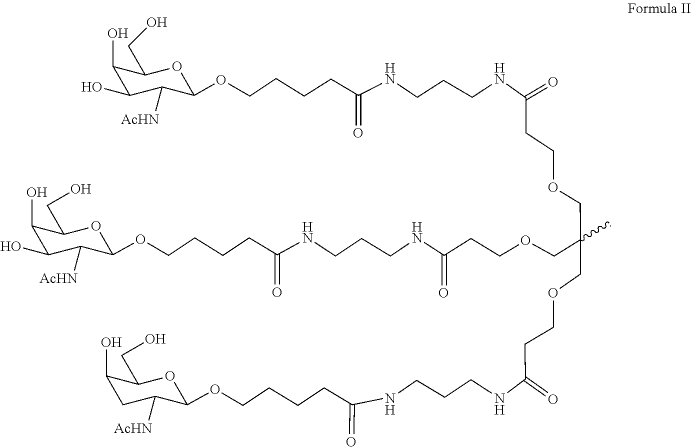 [SMILES: CC(=O)NC1CC(O)C(CO)OC1OCCCCC(=O)NCCCNC(=O)CCOCC(C)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, the siRNA agent is conjugated to L96 as defined in Table 1 and shown below

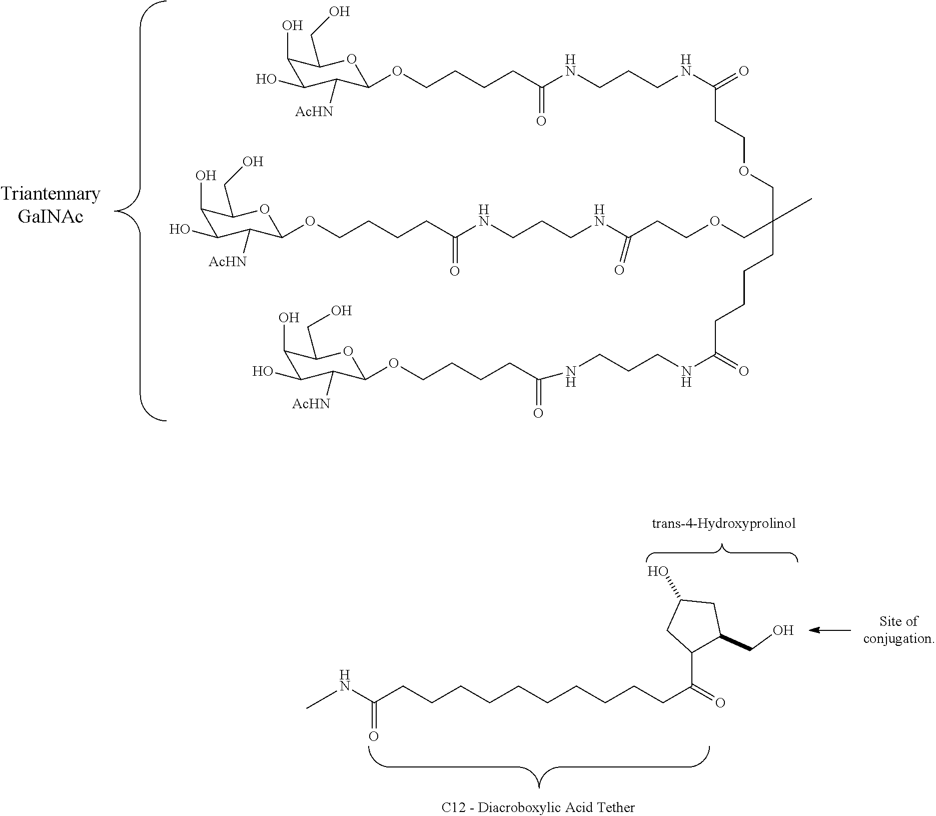

In certain embodiments, the target mRNA is chosen from a mammalian, plant, pathogen-associated, viral, or disease-associated mRNA. The target mRNA may be associated with a disease, e.g., a tumor-associated mRNA, or an autoimmune disease-associated mRNA. Exemplary target genes can be chosen from: Factor VII, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, HAMP, Activated Protein C gene, Cyclin D gene, VEGF gene, antithrombin 3 gene, aminolevulinate synthase 1 gene, alpha-1-antitrypsin gene, tmprss6 gene, apoal gene, apoc3 gene, bc11a gene, klf gene, angptl3 gene, plk gene, PKN3 gene, HBV, HCV, p53 gene, angiopoietin gene, or angiopoietin-like 3 gene. In certain embodiments, the target is chosen from: Eg5, PCSK9, TTR, HAMP, VEGF gene, antithrombin 3 gene, aminolevulinate synthase 1 gene, alpha-1-antitrypsin gene, tmprss6 gene, complement C5 gene, or complement C3 gene.

In certain embodiments, the nucleic acid molecules (e.g., double-stranded oligonucleotide or RNA molecule, e.g., dsRNA) described herein target a wild-type target RNA transcript variant, a mutant transcript, or a combination thereof. For example, the nucleic acid molecule can target a polymorphic variant, such as a single nucleotide polymorphism (SNP), of the target gene. In another embodiment, the nucleic acid molecule targets both a wildtype and a mutant target gene transcript. In other embodiments, the nucleic acid molecule targets a non-coding region of the target RNA transcript, such as the 5′ or 3′ untranslated region of a transcript.

### Immobilized Nucleic Acid Molecules

In certain embodiments, the nucleic acid molecule (e.g., an oligonucleotide molecule (e.g., a double-stranded oligonucleotide), or an RNA molecule, e.g., a double-stranded RNA (dsRNA)) is immobilized to a solid support, e.g., a surface, a plate or a bead. In embodiments, the immobilization of the nucleic acid molecule to the solid support provides one or more of a stable nucleic acid molecule, a qualitative display of the nucleic acid molecule, a quantitative display of the nucleic acid molecule, a substantially non-denatured nucleic acid molecule, or a nucleic acid molecule conformation that exposes one or more epitopes.

In certain embodiments, the nucleic acid molecule (e.g., an oligonucleotide molecule (e.g., a double-stranded oligonucleotide), or an RNA molecule, e.g., a double-stranded RNA (dsRNA)) is immobilized directly, e.g., covalently coupled, to the solid support. In one embodiment, the nucleic acid molecule comprises at least two strands (e.g., having a sense strand and an antisense strand). In some embodiments, the sense strand, the antisense strand, or both, is/are covalently coupled to the solid support. In one embodiment, the sense strand is immobilized to the solid support. In another embodiment, the antisense strand is immobilized to the solid support. In yet another embodiment, both the sense strand and the antisense strand are immobilized to the solid support. In some embodiments, the nucleic acid molecule (e.g., double-stranded oligonucleotide or RNA molecule, e.g., dsRNA) is covalently coupled to the solid support at one end of the strand (e.g., 5′ end and/or 3′ end). For example, the nucleic acid molecule (e.g., double-stranded oligonucleotide or RNA molecule, e.g., dsRNA) can be immobilized to the solid support at the 5′ end of the sense strand, 5′ end of the antisense strand, or both. In other embodiments, the nucleic acid molecule (e.g., double-stranded oligonucleotide or RNA molecule, e.g., dsRNA) is immobilized to the solid support at both ends of the molecule or strand (e.g., 5′ end and/or 3′ end). Exemplary orientations of the nucleic acid molecules (e.g., double-stranded oligonucleotide or RNA molecule, e.g., dsRNA) are depicted in FIG. 2B.

In one embodiment, the nucleic acid molecule (e.g., double-stranded oligonucleotide or RNA molecule, e.g., dsRNA) is phosphorylated at the 5′-end, e.g., the 5′-end of a sense or an antisense strand, or both. Exemplary phosphorylated configurations of an RNA molecule comprising a duplex of a GalNAc-conjugated sense strand and an antisense strand (AS) are depicted in FIG. 2A. The phosphorylated (e.g., 5′ phosphorylated) double stranded oligonucleotides or nucleic acid molecule can be immobilized to the solid support, e.g., a surface, plate or bead, coated with a reactive group. In one embodiment, the reactive group is chosen from an amine (e.g., secondary amino) group or a sulfhydryl group. In some embodiments, the phosphate group of the nucleic acid molecule (e.g., double-stranded oligonucleotide or RNA molecule, e.g., dsRNA) forms a covalent bond (e.g., a phosphoramidate bond) with the reactive group (e.g., the secondary amino group) present on the solid support, e.g., the surface of a plate. In certain embodiments, the nucleic acid molecule (e.g., double-stranded oligonucleotide or RNA molecule, e.g., dsRNA) is covalently coupled (e.g., through a phosphoramidate bond) to the solid support (e.g., a polystyrene surface, e.g., grafted with one or more secondary amino groups).

The density of the reactive group on the plate may vary. In certain embodiments, the density of the reactive group is between about 10¹⁰/cm² and about 10¹⁶/cm², e.g., between about 10¹²/cm² and about 10¹⁴/cm², e.g., about 10¹²/cm², about 10¹³/cm², about 10¹⁴/cm², about 10¹⁵/cm², or about 10¹⁶/cm².

The reactive group may optionally comprise a linker. In certain embodiments, the linker includes a spacer arm that is covalently grated to the plate surface. The reactive group can be positioned at the end of the spacer arm as depicted in FIG. 3.

In other embodiments, the nucleic acid molecule (e.g., double-stranded oligonucleotide or RNA molecule, e.g., dsRNA) is immobilized via a non-covalent (e.g., affinity) interaction to the solid support. In one embodiment, the nucleic acid molecule (e.g., double-stranded oligonucleotide or RNA molecule, e.g., dsRNA) is immobilized to the solid support via an antigen-antibody interaction. For example, the plate can be coated with an antibody molecule to the nucleic acid molecule (e.g., an antibody molecule as described herein) such that the nucleic acid molecule can be immobilized to the plate through the antigen-antibody interaction.

In other embodiments, the nucleic acid molecule (e.g., double-stranded oligonucleotide or RNA molecule, e.g., dsRNA) is immobilized to the solid support via an affinity agent that interacts with a partner moiety coupled to the nucleic acid molecule. Exemplary affinity agents include a protein or ligand of a protein-ligand pair, e.g., biotin-streptavidin. In one embodiment, the solid surface, e.g., plate, is be coated with streptavidin such that a biotinylated nucleic acid molecule can be immobilized to the plate through the streptavidin-biotin affinity interaction.

Various types of solid supports, e.g., plates, can be coated with the nucleic acid molecule (e.g., double-stranded oligonucleotide or RNA molecule, e.g., dsRNA) or non-covalent partner. Suitable solid phase supports include any support capable of binding a nucleic acid, a protein or an antibody. Exemplary supports include glass, polystyrene, polypropylene, polyethylene, dextran, nylon, amylases, natural and modified celluloses, polyacrylamides, gabbros, and magnetite. In one embodiment, the solid support is polystyrene. For example, a plate (e.g., a polystyrene plate) can be grafted with a reactive group, e.g., an amine (e.g., a secondary amino) group or a sulfhydryl group as described herein. In one embodiment, the plate is coated with a secondary amino group (e.g., a CovaLink™ NH plate). In another embodiment, the plate is a maleimide activated plate. In yet another embodiment, the plate is coated with streptavidin.

### Detection

In certain embodiments, the detection or determining steps of the methods, assays, kits described herein include determining qualitatively or quantitatively the value (e.g., level, e.g., amount or concentration) of the antibody (e.g., ADA) against the nucleic acid molecule (e.g., an oligonucleotide molecule (e.g., a double-stranded oligonucleotide), or an RNA molecule, e.g., a double-stranded RNA (dsRNA)). In certain embodiments, the antibody (e.g., ADA) is present in a sample, e.g., a sample of plasma, serum, blood, or other non-cellular body fluid, wherein the amount or concentration of the antibody (e.g., ADA) provides a value. In certain embodiments, the determined or detected value is compared to a specified parameter (e.g., a reference value; a control sample; a sample obtained from a healthy subject; a sample acquired from the subject at different time intervals, e.g., prior to, during, or after a treatment); or a value acquired using a positive or negative control, e.g., a positive control antibody as described herein. In certain embodiments, treatment includes administration of a nucleic acid molecule, e.g., a nucleic acid described herein.

In certain embodiments, the detection step comprises a colorimetric means for evaluating the level of the anti- double stranded oligonucleotides or anti-nucleic acid molecule antibody or ADA. Exemplary colorimetric means can be chosen from absorbance, fluorescent intensity or polarization.

In certain embodiments, a detection agent is used in the methods, assays and kits described herein that specifically binds to the complex of the anti-nucleic acid molecule antibody and the immobilized nucleic acid molecule. In one embodiment, the detection agent is a detection antibody that binds to the antibody that binds to the nucleic acid molecule (e.g., the

ADA) present in the sample. For example, the detection antibody binds to an IgA, IgE, IgG or an IgM (e.g., a human IgG or an IgM), or a portion thereof, e.g., an Fc region of an IgG or an IgM.

In another embodiment, the detection agent, e.g., the detection antibody, is detectably labeled. In one embodiment, the detectable labeled agent, e.g., antibody, is chosen from a radiolabeled, a chromophore-labeled, a fluorophore-labeled, or an enzyme-labeled. In one embodiment, the agent is an antibody derivative (e.g., an antibody or antibody fragment conjugated with a substrate, or with the protein or ligand of a protein-ligand pair, e.g., biotin-streptavidin. In one embodiment, binding of the detection agent, e.g., the detection antibody, to the complex is detected using an antibody conjugated to an enzyme, a prosthetic group complex, a fluorescent material, a luminescent material, or a radioactive material. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials include, but are not limited to, ¹²⁵I, ¹³¹I, ³⁵S or ³H. In one embodiment, the antibody conjugated to an enzyme such as peroxidase that can catalyze a color-producing reaction. Alternatively, the antibody can also be tagged to a fluorophore, such as fluorescein, rhodamine, DyLight Fluor or Alexa Fluor. In such embodiments, the detection is usually carried out by a floursecent molecule bound to the detection agent, e.g., detection, antibody by biotin.

### Samples and Subjects

In certain embodiments, the method, or assay, further includes the step of acquiring a sample, e.g., a biological sample, from a subject. In one embodiment, the method, or assay, includes the step of obtaining a predominantly non-cellular fraction of a body fluid from the subject. The non-cellular fraction can be plasma, serum, or other non-cellular body fluid. In one embodiment, the sample is a serum sample. In other embodiments, the body fluid from which the sample is obtained from an individual comprises blood (e.g., whole blood). In certain embodiments, the blood can be further processed to obtain plasma or serum.

For any of the methods or assays disclosed herein, the subject from which the sample is acquired, has undergone, is undergoing or will receive a treatment that comprises the nucleic acid molecule. In certain embodiments, the nucleic acid molecule targets an mRNA that may be associated with a disease, e.g., a tumor-associated mRNA, or an autoimmune disease-associated mRNA. Exemplary target genes can be chosen from: Factor VII, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, HAMP, Activated Protein C gene, Cyclin D gene, VEGF gene, antithrombin 3 gene, aminolevulinate synthase 1 gene, alpha-1-antitrypsin gene, tmprss6 gene, apoal gene, apoc3 gene, be11a gene, klf gene, angptl3 gene, plk gene, PKN3 gene, HBV, HCV, p53 gene, angiopoietin gene, or angiopoietin-like 3 gene. In certain embodiments, the target is chosen from: Eg5, PCSK9, TTR, HAMP, VEGF gene, antithrombin 3 gene, aminolevulinate synthase 1 gene, alpha-1-antitrypsin gene, tmprss6 gene, complement C5 gene, or complement C3 gene.

The methods described herein can further include the step of monitoring the subject, e.g., for a change (e.g., an increase or decrease) in an ADA response (e.g., using the methods and assays described herein). Further parameters related to clinical response that can be evaluated include, but are not limited to, a hypersensitivity response, autoimmunity, pharmacokinetics, drug neutralization, abnormal biodistribution, and/or enhanced drug clearance rates. The subject can be monitored in one or more of the following periods: prior to beginning of treatment; during the treatment; or after the treatment has been administered.

In other embodiments, the methods, assays, and/or kits described herein further include providing or generating, and/or transmitting information, e.g., a report, containing data of the evaluation or treatment determined by the methods, assays, and/or kits as described herein. The information can be transmitted to a report-receiving party or entity (e.g., a patient, a health care provider, a diagnostic provider, and/or a regulatory agency, e.g., the FDA), or otherwise submitting information about the methods, assays and kits disclosed herein to another party. The method can relate to compliance with a regulatory requirement, e.g., a pre- or post approval requirement of a regulatory agency, e.g., the FDA. In one embodiment, the report-receiving party or entity can determine if a predetermined requirement or reference value is met by the data, and, optionally, a response from the report-receiving entity or party is received, e.g., by a physician, patient, diagnostic provider.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### BRIEF DESCRIPTION OF THE DRAWINGS

FIG. 1 is a schematic representation of an ELISA assay showing direct immobilization, e.g., coupling, of a double stranded oligonucleotides or nucleic acid molecule to a solid support (1). In this representation, the double stranded oligonucleotides or nucleic acid molecule is a GalNAc-siRNA conjugate (GalNac is depicted as a solid circle attached to a line, which represents the iRNA). Upon binding of an anti- double stranded oligonucleotides or anti-nucleic acid antibody to the immobilized double stranded oligonucleotides or nucleic acid molecule, a complex is formed (2). The formation of the complex is detected using a detection reagent, for example, a labeled-conjugated secondary antibody, e.g., an anti-IgG/M coupled to a horseradish peroxidase (HRP) detection moiety (3).

FIG. 2A is a schematic representation of the phosphorylation of the siRNA duplex, sense strand, and antisense strand. The sense strand of the siRNA duplex contains a GalNAc moiety at the 3′ end.

FIG. 2B shows how a phosphorylated siRNA conjugate can be covalently coupled to the plate through the 5′ phosphate group(s) of the duplex. The phosphorylated siRNA conjugate can be covalently coupled to the plate at the 5′ end of the sense strand, 5′ end of the antisense strand, or both.

FIG. 3 depicts the structure of the linkers grafted onto the surface of the plate. The linker contains a secondary amino group positioned at the end of the spacer arm.

FIG. 4 is a schematic representation of the coupling reaction between the 5′ phosphate group of the siRNA conjugate and the secondary amino group positioned at the end of the spacer arm covalently grafted to the polystyrene surface.

FIG. 5A depicts the results of RT-qPCR indicating the amount (pg) of AD-59153 (having 5′ phosphate in the sense strand) coupled in each well. The results for each individual experiment, either in the absence of EDC (at 50° C.) or in the present EDC (at 50° C. or 37° C.), were shown.

FIG. 5B depicts the average amount (pg) of coupled AD-59153 based on the results shown in FIG. 5A.

FIG. 6A depicts the results of RT-qPCR indicating the amount (pg) of AD-59155 (having 5′ phosphate in the sense strand) coupled in each well. The results for each individual experiment, either in the absence of EDC or in the present EDC, were shown.

FIG. 6B depicts the average amount (pg) of coupled AD-59155 based on the results shown in FIG. 6A.

FIG. 7A is another example showing the amount (pg) of AD-59155 coated per well.

FIG. 7B depicts the results of ELISA using serial dilutions of the anti-AD-59155 serum from rabbit #18273 on Day 110 to evaluate various HRP conjugated secondary antibodies.

FIG. 8 depicts the results of ELISA using the anti-KLH-AD-59153 serum from rabbit #19151, Day 42, serially diluted in either blocking buffer (casein/TBS) or pooled human sera (1/50 in blocking buffer).

FIG. 9A depicts the results of ELISA performed in the plate coated with AD-59153, using serially diluted anti-KLH-AD-59153 serum from rabbit 19151, Day 42, or pre-bleed serum from the same rabbit.

FIG. 9B depicts the results of ELISA performed in the drug-free plate, or the plates coated with AD-59153, AD-59155, or AD-57740 (Luc) using the anti-KLH- AD-59153 serum from rabbit 19151, Day 42.

FIG. 9C depicts the correlation between the binding of the anti- AD-59153 antibodies to AD-59153 coated plate and the amount of AD-59153.

FIG. 10A depicts the results of ELISA performed in the uncoated plate or the plate coated with AD-59155 using the anti-KLH-AD-59155 serum from rabbit #19180, Day 42.

FIG. 10B depicts the correlation between the binding of the anti-AD-59155 antibodies to AD-59155 coated plate and the amount of AD-59155.

FIG. 11A depicts the results of ELISA performed in the plates coated with various AD-59155, AD-59153, or control compounds using the polyclonal anti-AD-59155 antibodies from rabbit #18273 (Day 139).

FIG. 11B depicts the results of ELISA performed in the plates coated with various AD-59155, AD-59153, or control compounds using the polyclonal anti-AD-59155 antibodies from rabbit #19178 (Day 70) (right bars), or pre-bleed serum (left bars).

FIG. 12 depicts the results of ELISA performed in the plates coated with various AD-59155, AD-59153, or control compounds using the polyclonal anti-AD-59153 antibodies from rabbit #19151 (Day 98) (right bars), or pre-bleed serum (left bars).

### DETAILED DESCRIPTION

The induction of anti-drug antibodies (ADAs) can result in adverse clinical results such as hypersensitivity and autoimmunity, as well as altered pharmacokinetics (e.g., drug neutralization, abnormal biodistribution, and enhanced drug clearance rates). These clinical results can alter the efficacy of a drug treatment. Therefore, immune responses caused by drug therapeutics can be an important safety and efficacy concern for regulatory agencies, drug manufacturers, clinicians, and patients.

Accordingly, assays, methods, reagents and kits for evaluating, e.g., detecting the level of, an antibody against a nucleic acid molecule (e.g., an oligonucleotide molecule (e.g., a double-stranded oligonucleotide), or an RNA molecule, e.g., a double-stranded RNA (dsRNA)) (e.g., an anti-drug antibody (ADA)) are disclosed. Additionally disclosed herein are binding agents that can be used in the detection, calibration and/or quantification of the antibodies or the nucleic acid molecules (e.g., double-stranded oligonucleotide or RNA molecule, e.g., dsRNA). In one embodiment, the binding agent is an antibody molecule that binds to the nucleic acid molecule.

In one embodiment, the nucleic acid molecule (e.g., double-stranded oligonucleotide or RNA molecule, e.g., dsRNA) is immobilized, e.g., directly or indirectly, to a solid support. For example, Applicants have discovered that immobilization, e.g., covalent immobilization, of a nucleic acid molecule (e.g., double-stranded oligonucleotide or RNA molecule, e.g., dsRNA) to a solid support provides a stable, qualitative and quantifiable display of a substantially non-denatured double stranded oligonucleotides or nucleic acid molecule. In another embodiment, the nucleic acid molecule (e.g., double-stranded oligonucleotide or RNA molecule, e.g., dsRNA) is immobilized via a binding agent, e.g., an antibody molecule, to a solid support. Contacting of the immobilized nucleic acid molecule with a sample (e.g., a plasma sample, a serum sample or a whole blood sample from a subject) can be effected under conditions that allow binding of an antibody against the nucleic acid molecule (an “anti-nucleic acid molecule antibody”), if present in the sample, to the immobilized nucleic acid molecule, thereby forming a complex between the anti-nucleic acid molecule antibody and the immobilized nucleic acid molecule. Optionally, a detection agent that specifically binds to the complex of the anti-nucleic acid molecule antibody and the immobilized nucleic acid molecule can be added, thereby allowing detection of the bound anti-nucleic acid molecule antibody, if present in the sample.

The contacting step is effected in a solid support, e.g., using an enzyme-linked immunosorbent assay (ELISA). Alternative exemplary ELISA formats described herein, include, but are not limited to, indirect ELISA, sandwich ELISA, competitive ELISA, and multiple and portable ELISA.

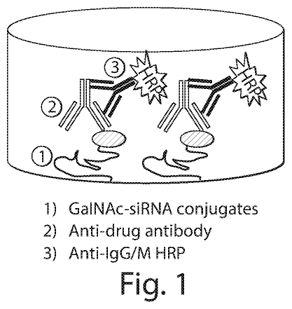

One embodiment of an ELISA assay is summarized as FIG. 1. FIG. 1 provides a schematic representation of an ELISA assay showing direct immobilization, e.g., coupling, of a nucleic acid molecule (e.g., double-stranded oligonucleotide or RNA molecule, e.g., dsRNA) to a solid support (1). In this representation, the nucleic acid molecule is a GalNAc-siRNA conjugate (GalNac is depicted as a solid circle attached to a line, which represents the iRNA).

Upon binding of an anti-nucleic acid antibody to the immobilized nucleic acid molecule, a complex is formed (2). The formation of the complex is detected using a detection reagent, in this case, a labeled-conjugated secondary antibody, e.g., an anti-IgG/M coupled to a horseradish peroxidase (HRP) detection moiety (3).

Alternatively described herein are embodiments where the contacting step is effected in solution, e.g., using a radioimmunoassay (RIA).

Various aspects of the invention are described in further detail in the following subsections.

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“G,” “C,” “A,” “T” and “U” each generally stand for a nucleotide that contains guanine, cytosine, adenine, thymidine and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the assays, methods, compositions, and kits featured in the invention.

As used herein, the term “iRNA,” “RNAi”, “iRNA agent,” or “RNAi agent” refers to an agent that contains RNA as that term is defined herein, and which mediates the targeted cleavage of an RNA transcript, e.g., via an RNA-induced silencing complex (RISC) pathway. In one embodiment, an iRNA as described herein effects inhibition of TTR expression. Inhibition of target gene expression may be assessed based on a reduction in the level of target gene mRNA or a reduction in the level of the target gene protein. As used herein, “target sequence” refers to a contiguous portion of the nucleotide sequence of an mRNA molecule formed during the transcription of a target gene, including mRNA that is a product of RNA processing of a primary transcription product. The target portion of the sequence will be at least long enough to serve as a substrate for iRNA-directed cleavage at or near that portion. For example, the target sequence will generally be from 9-36 nucleotides in length, e.g., 15-30 nucleotides in length, including all sub-ranges therebetween. As non-limiting examples, the target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides,20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they may form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing.

The terms “complementary,” “fully complementary” and “substantially complementary” herein may be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding a target gene protein). For example, a polynucleotide is complementary to at least a part of a target gene mRNA if the sequence is substantially complementary to a non-interrupted portion of an mRNA encoding target gene. As another example, a polynucleotide is complementary to at least a part of a target gene mRNA if the sequence is substantially complementary to a non-interrupted portion of an mRNA encoding target gene.

The term “double-stranded oligonucleotide” as used herein, refers to an oligonucleotide that includes a DNA molecule (e.g., deoxyribonucleoside-containing molecule) or an RNA molecule (e.g., ribonucleoside-containing molecule), or a combination of DNA/RNA molecule, having a hybridized duplex region that comprises two anti-parallel and substantially complementary nucleic acid strands, which will be referred to as having “sense” and “antisense” orientations with respect to a target nucleotide sequence, e.g., RNA. In certain embodiments, the double-stranded oligonucleotide includes one or more deoxyribonucleosides and one or more ribonucleoside in one or both strands of the molecule.

The term “double-stranded RNA” or “dsRNA,” as used herein, refers to an iRNA that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA, e.g., through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. dsRNAs generated in the cell by processing with Dicer and similar enzymes are generally in the range of 19-22 base pairs in length. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA as described above.

In another embodiment, the iRNA agent may be a “single-stranded siRNA” that is introduced into a cell or organism to inhibit a target mRNA. Single-stranded RNAi agents bind to the RISC endonuclease Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150: 883-894, the entire contents of each of which are hereby incorporated herein by reference. Any of the antisense nucleotide sequences described herein (e.g., sequences provided in Table 2) may be used as a single-stranded siRNA as described herein or as chemically modified by the methods described in Lima et al., (2012) Cell 150:883-894.

In another aspect, the RNA agent is a “single-stranded antisense RNA molecule.” An single-stranded antisense RNA molecule is complementary to a sequence within the target mRNA. Single-stranded antisense RNA molecules can inhibit translation in a stoichiometric manner by base pairing to the mRNA and physically obstructing the translation machinery, see Dias, N. et al., (2002) Mol Cancer Ther 1:347-355. Alternatively, the single-stranded antisense molecules inhibit a target mRNA by hydridizing to the target and cleaving the target through an RNaseH cleavage event. The single-stranded antisense RNA molecule may be about 10 to about 30 nucleotides in length and have a sequence that is complementary to a target sequence. For example, the single-stranded antisense RNA molecule may comprise a sequence that is at least about 10, 11, 12, 13, 14, 15, 16, 17, 18, 19, 20, or more contiguous nucleotides from any one of the antisense nucleotide sequences described herein, e.g., sequences provided in Table 2.

The term “nucleic acid molecule” encompasses an RNA molecule (e.g., an RNA molecule as described herein), a DNA molecule (e.g., a 100% deoxynucleoside-containing molecule), and a combination of an RNA and a DNA molecule. It includes a naturally-occurring and non-naturally-occurring nucleic acid molecule. In one embodiment, the nucleic acid molecule is isolated or purified. In one embodiment, the nucleic acid molecule is synthetic (e.g., chemically synthesized) or recombinant. In other embodiments, the nucleic acid molecule is a non-naturally-occurring nucleic acid molecule, e.g., an analog or a derivative of a nucleic acid molecule, e.g., analogs and derivatives of DNA, RNA or both. For example, the nucleic acid molecule can include one or more nucleotide/nucleoside analogs or derivatives as described herein or as known in the art. In certain embodiments, “nucleic acid molecule” includes an oligonucleotide molecule (e.g., a single-stranded or a double-stranded oligonucleotide (e.g., an oligodeoxyribonucleotide or an oligoribonucleotide, or a combination thereof)). In other embodiments, “nucleic acid molecule” includes an RNA molecule, e.g., a single-stranded or a double-stranded RNA (dsRNA), e.g., as described herein. In certain embodiments, the nucleic acid molecule comprises at least 5, 10, 15, 20, 25, 30, 35, 40, 45, 50, 55, 60, 65, 70, 75, 80, 85, 90, 95% or 100% deoxyribonucleosides, e.g., in one or both strands.

The term “RNA molecule” or “ribonucleic acid molecule” encompasses a naturally-occurring and non-naturally-occurring RNA molecule. In one embodiment, the RNA molecule is isolated or purified. In one embodiment, the RNA molecule is synthetic (e.g., chemically synthesized) or recombinant. In other embodiments, the RNA molecule is a non-naturally-occurring RNA molecule, e.g., an analog or a derivative of an RNA molecule. In certain embodiments, the RNA molecule comprises one or more ribonucleotide/ribonucleoside analogs or derivatives as described herein or as known in the art. A “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The ribonucleoside or ribonucleotide can be modified in the nucleobase structure or in the ribose-phosphate backbone structure, e.g., as described herein below. In certain embodiments, the RNA molecule that comprises a ribonucleoside analog or derivative retains the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an RNA molecule can comprise at least two modified ribonucleosides, at least 3, at least 4, at least 5, at least 6, at least 7, at least 8, at least 9, at least 10, at least 15, at least 20 or more, up to the entire length of the RNA molecule. The modifications need not be the same for each of such a plurality of modified ribonucleosides in an RNA molecule. In one embodiment, modified RNA molecules contemplated for use in methods and compositions described herein include peptide nucleic acids (PNAs) that have the ability to form the required duplex structure and that permit or mediate the specific degradation of a target RNA, e.g., via a RISC pathway.

Exemplary RNA molecules, include but are not limited to, iRNA agents or molecules, double stranded RNA (dsRNA) molecules, siRNA molecules, single-stranded RNAi molecules, single-stranded siRNA molecules, microRNA (miRNA), antisense RNA, short hairpin RNA (shRNA), antagomirs, mRNA, decoy RNA, vectors and aptamers.

In certain embodiments, an RNA molecule comprises a deoxyribonucleoside. For example, the RNA molecule, e.g., an iRNA agent can comprise one or more deoxynucleosides, including, for example, a deoxynucleoside overhang(s), or one or more deoxynucleosides within the double stranded portion of a dsRNA. In certain embodiments, the RNA molecule comprises a percentage of deoxyribonucleosides of at least 5, 10, 15, 20, 25, 30, 35, 40, 45, 50, 55, 60, 65, 70, 75, 80, 85, 90, 95% or higher (but not 100%) deoxyribonucleosides, e.g., in one or both strands. In certain embodiments, the term “iRNA” does not encompass a double stranded DNA molecule (e.g., a naturally-occurring double stranded DNA molecule or a 100% deoxynucleoside-containing DNA molecule).

In one aspect, an RNA interference agent includes a single stranded RNA that interacts with a target RNA sequence to direct the cleavage of the target RNA. Without wishing to be bound by theory, long double stranded RNA introduced into cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188). Thus, in one aspect the invention relates to a single stranded RNA that promotes the formation of a RISC complex to effect silencing of the target gene.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of a dsRNA.

In one embodiment, the antisense strand of a dsRNA has a 1-10 nucleotide overhang at the 3′ end and/or the 5′ end. In one embodiment, the sense strand of a dsRNA has a 1-10 nucleotide overhang at the 3′ end and/or the 5′ end. In another embodiment, one or more of the nucleotides in the overhang is replaced with a nucleoside thiophosphate.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double-stranded over its entire length.

The term “antisense strand” or “guide strand” refers to the strand of an iRNA, e.g., a dsRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches may be in the internal or terminal regions of the molecule. Generally, the most tolerated mismatches are in the terminal regions, e.g., within 5, 4, 3, or 2 nucleotides of the 5′ and/or 3′ terminus.

The term “sense strand,” or “passenger strand” as used herein, refers to the strand of an iRNA that includes a region that is substantially complementary to a region of the antisense strand as that term is defined herein.

As used herein, the term “SNALP” refers to a stable nucleic acid-lipid particle. A SNALP represents a vesicle of lipids coating a reduced aqueous interior comprising a nucleic acid such as an iRNA or a plasmid from which an iRNA is transcribed. SNALPs are described, e.g., in U.S. Patent Application Publication Nos. 20060240093, 20070135372, and in International Application No. WO 2009082817. These applications are incorporated herein by reference in their entirety.

“Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA may also be “introduced into a cell,” wherein the cell is part of a living organism. In such an instance, introduction into the cell will include the delivery to the organism. For example, for in vivo delivery, iRNA can be injected into a tissue site or administered systemically. In vivo delivery can also be by a β-glucan delivery system, such as those described in U.S. Pat. Nos. 5,032,401 and 5,607,677, and U.S. Publication No. 2005/0281781, which are hereby incorporated by reference in their entirety. In vitro introduction into a cell includes methods known in the art such as electroporation and lipofection. Further approaches are described herein below or known in the art.

As used herein, the term “modulate the expression of,” refers to at an least partial “inhibition” or partial “activation” of a target gene expression in a cell treated with an iRNA composition as described herein compared to the expression of target gene in a control cell. A control cell includes an untreated cell, or a cell treated with a non-targeting control iRNA.

The terms “activate,” “enhance,” “up-regulate the expression of,” “increase the expression of,” and the like, in so far as they refer to a target gene, herein refer to the at least partial activation of the expression of a target gene, as manifested by an increase in the amount of target gene mRNA, which may be isolated from or detected in a first cell or group of cells in which a target gene is transcribed and which has or have been treated such that the expression of a target gene is increased, as compared to a second cell or group of cells substantially identical to the first cell or group of cells but which has or have not been so treated (control cells).

In one embodiment, expression of a target gene is activated by at least about 10%, 15%, 20%, 25%, 30%, 35%, 40%, 45%, or 50% by administration of an iRNA as described herein. In some embodiments, a target gene is activated by at least about 60%, 70%, or 80% by administration of an iRNA featured in the invention. In some embodiments, expression of a target gene is activated by at least about 85%, 90%, or 95% or more by administration of an iRNA as described herein. In some embodiments, the target gene expression is increased by at least 1-fold, at least 2-fold, at least 5-fold, at least 10-fold, at least 50-fold, at least 100-fold, at least 500-fold, at least 1000 fold or more in cells treated with an iRNA as described herein compared to the expression in an untreated cell. Activation of expression by small dsRNAs is described, for example, in Li et al., 2006 Proc. Natl. Acad. Sci. U.S.A. 103:17337-42, and in US20070111963 and US2005226848, each of which is incorporated herein by reference.

The terms “silence,” “inhibit expression of,” “down-regulate expression of,” “suppress expression of,” and the like, in so far as they refer to a target gene, herein refer to the at least partial suppression of the expression of a target gene, as assessed, e.g., based on target gene mRNA expression, target gene protein expression, or another parameter functionally linked to target gene expression. For example, inhibition of target gene expression may be manifested by a reduction of the amount of target gene mRNA which may be isolated from or detected in a first cell or group of cells in which a target gene is transcribed and which has or have been treated such that the expression of a target gene is inhibited, as compared to a control. The control may be a second cell or group of cells substantially identical to the first cell or group of cells, except that the second cell or group of cells have not been so treated (control cells). The degree of inhibition is usually expressed as a percentage of a control level, e.g.,

$$\frac{( mRNA in control cells ) - ( mRNA in treated cells )}{( mRNA in control cells )} •100 \%$$

Alternatively, the degree of inhibition may be given in terms of a reduction of a parameter that is functionally linked to target gene expression, e.g., the amount of protein encoded by a target gene. The reduction of a parameter functionally linked to target gene expression may similarly be expressed as a percentage of a control level. In principle, target gene silencing may be determined in any cell expressing target gene, either constitutively or by genomic engineering, and by any appropriate assay. However, when a reference is needed in order to determine whether a given iRNA inhibits the expression of the target gene by a certain degree and therefore is encompassed by the instant invention, the assays provided in the Examples below shall serve as such reference.

For example, in certain instances, expression of a target gene is suppressed by at least about 10%, 15%, 20%, 25%, 30%, 35%, 40%, 45%, or 50% by administration of an iRNA featured in the invention. In some embodiments, a target gene is suppressed by at least about 60%, 65%, 70%, 75%, or 80% by administration of an iRNA featured in the invention. In some embodiments, a target gene is suppressed by at least about 85%, 90%, 95%, 98%, 99%, or more by administration of an iRNA as described herein.

As used herein in the context of target gene expression, the terms “treat,” “treating,” “treatment,” and the like, refer to relief from or alleviation of pathological processes related to target gene expression. In the context of the present invention insofar as it relates to any of the other conditions recited herein below (other than pathological processes related to target gene expression), the terms “treat,” “treatment,” and the like mean to prevent, relieve or alleviate at least one symptom associated with such condition, or to slow or reverse the progression or anticipated progression of such condition. Thus, unless the context clearly indicates otherwise, the terms “treat,” “treatment,” and the like are intended to encompass prophylaxis, e.g., prevention of disorders and/or symptoms of disorders related to target gene expression.

By “lower” in the context of a disease marker or symptom is meant a statistically or clinically significant decrease in such level. The decrease can be, for example, at least 10%, at least 20%, at least 30%, at least 40% or more, and is typically down to a level accepted as within the range of normal for an individual without such disorder.

As used herein, the phrases “therapeutically effective amount” and “prophylactically effective amount” refer to an amount that provides a therapeutic benefit in the treatment, prevention, or management of pathological processes related to target gene expression. The specific amount that is therapeutically effective can be readily determined by an ordinary medical practitioner, and may vary depending on factors known in the art, such as, for example, the type of pathological process, the patient's history and age, the stage of pathological process, and the administration of other agents.

As used herein, a “pharmaceutical composition” comprises a pharmacologically effective amount of an iRNA and a pharmaceutically acceptable carrier. As used herein, “pharmacologically effective amount,” “therapeutically effective amount” or simply “effective amount” refers to that amount of an iRNA effective to produce the intended pharmacological, therapeutic or preventive result. For example, in a method of treating a disorder related to target gene expression, an effective amount includes an amount effective to reduce one or more symptoms associated with the disease, or an amount effective to reduce the risk of developing conditions associated with the disease. For example, if a given clinical treatment is considered effective when there is at least a 10% reduction in a measurable parameter associated with a disease or disorder, a therapeutically effective amount of a drug for the treatment of that disease or disorder is the amount necessary to effect at least a 10% reduction in that parameter. For example, a therapeutically effective amount of an iRNA targeting target gene can reduce target gene protein levels by any measurable amount, e.g., by at least 10%, 20%, 30%, 40% or 50%.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Agents included in drug formulations are described further herein below.

The term “about” when referring to a number or a numerical range means that the number or numerical range referred to is an approximation within experimental variability (or within statistical experimental error), and thus the number or numerical range may vary from, for example, between 1% and 15% of the stated number or numerical range.

“Acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Sample,” “tissue sample,” “patient sample,” “patient cell or tissue sample” or “specimen” each refers to a biological sample obtained from a tissue or bodily fluid of a subject or patient. The source of the tissue sample can be solid tissue as from a fresh, frozen and/or preserved organ, tissue sample, biopsy, or aspirate; blood or any blood constituents (e.g., serum, plasma); bodily fluids such as cerebral spinal fluid, whole blood, plasma and serum. The sample can include a non-cellular fraction (e.g., plasma, serum, or other non-cellular body fluid). In one embodiment, the sample is a serum sample. In other embodiments, the body fluid from which the sample is obtained from an individual comprises blood (e.g., whole blood). In certain embodiments, the blood can be further processed to obtain plasma or serum. In another embodiment, the sample contains a tissue, cells (e.g., peripheral blood mononuclear cells (PBMC)). For example, the sample can be a fine needle biopsy sample, an archival sample (e.g., an archived sample with a known diagnosis and/or treatment history), a histological section (e.g., a frozen or formalin-fixed section, e.g., after long term storage), among others. The term sample includes any material obtained and/or derived from a biological sample, including a polypeptide, and nucleic acid (e.g., genomic DNA, cDNA, RNA) purified or processed from the sample. Purification and/or processing of the sample can involve one or more of extraction, concentration, antibody isolation, sorting, concentration, fixation, addition of reagents and the like. The sample can contain compounds that are not naturally intermixed with the tissue in nature such as preservatives, anticoagulants, buffers, fixatives, nutrients, antibiotics or the like.

Double-Stranded Oligonucleotides or Ribonucleic Acid (dsRNA)

Described herein are iRNA agents that inhibit the expression of a target gene. In one embodiment, the iRNA agent includes double-stranded ribonucleic acid (dsRNA) molecules for inhibiting the expression of a target gene in a cell or in a subject (e.g., in a mammal, e.g., in a human), where the dsRNA includes an antisense strand having a region of complementarity which is complementary to at least a part of an mRNA formed in the expression of a target gene, and where the region of complementarity is 30 nucleotides or less in length, generally 19-24 nucleotides in length, and where the dsRNA, upon contact with a cell expressing the target gene, inhibits the expression of the target gene by at least 10% as assayed by, for example, a PCR or branched DNA (bDNA)-based method, or by a protein-based method, such as by Western blot. In one embodiment, the iRNA agent activates the expression of a target gene in a cell or mammal. Expression of a target gene in cell culture, such as in COS cells, HeLa cells, primary hepatocytes, HepG2 cells, primary cultured cells or in a biological sample from a subject can be assayed by measuring target gene mRNA levels, such as by bDNA or TaqMan assay, or by measuring protein levels, such as by immunofluorescence analysis, using, for example, Western Blotting or flow cytometric techniques.

A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence, derived from the sequence of an mRNA formed during the expression of a target gene. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, e.g., 15-30 nucleotides in length.

One of skill in the art will also recognize that the duplex region is a primary functional portion of a dsRNA, e.g., a duplex region of 9 to 36, e.g., 15-30 base pairs. Thus, in one embodiment, to the extent that it becomes processed to a functional duplex of e.g., 15-30 base pairs that targets a desired RNA for cleavage, an RNA molecule or complex of RNA molecules having a duplex region greater than 30 base pairs is a dsRNA. Thus, an ordinarily skilled artisan will recognize that in one embodiment, then, a miRNA is a dsRNA. In another embodiment, a dsRNA is not a naturally occurring miRNA. In another embodiment, an siRNA agent useful to target target gene expression is not generated in the target cell by cleavage of a larger dsRNA.

A dsRNA as described herein may further include one or more single-stranded nucleotide overhangs. The dsRNA can be synthesized by standard methods known in the art as further discussed below, e.g., by use of an automated DNA synthesizer, such as are commercially available from, for example, Biosearch, Applied Biosystems, Inc. In one embodiment, a target gene is a human target gene. In another embodiment the target gene is a mouse or a rat target gene. In specific embodiments, the first sequence is a sense strand of a dsRNA that includes a sense sequence, and the second sequence is an antisense strand of a dsRNA that includes an antisense sequence. Alternative dsRNA agents that target sequences other than those of the dsRNAs disclosed herein can readily be determined using the target sequence and the flanking target gene sequence.

The skilled person is well aware that dsRNAs having a duplex structure of between 20 and 23, but specifically 21, base pairs have been hailed as particularly effective in inducing RNA interference (Elbashir et al., EMBO 2001, 20:6877-6888). However, others have found that shorter or longer RNA duplex structures can be effective as well. In the embodiments described above, dsRNAs described herein can include at least one strand of a length of minimally 21 nucleotides. It can be reasonably expected that shorter duplexes minus only a few nucleotides on one or both ends may be similarly effective as compared to the dsRNAs described above. Hence, dsRNAs having a partial sequence of at least 15, 16, 17, 18, 19, 20, or more contiguous nucleotides, and differing in their ability to inhibit the expression of a target gene by not more than 5, 10, 15, 20, 25, or 30% inhibition from a dsRNA comprising the full sequence, are contemplated according to the invention.

In addition, the RNAs identify a site in a target gene transcript that is susceptible to RISC-mediated cleavage. As such, the present invention further features siRNAs that target within one of such sequences. As used herein, an siRNA is said to target within a particular site of an RNA transcript if the siRNA promotes cleavage of the transcript anywhere within that particular site. Such an siRNA will generally include at least 15 contiguous nucleotides coupled to additional nucleotide sequences taken from the region contiguous to the selected sequence in a target gene.

While a target sequence is generally 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that may serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an siRNA agent, mediate the best inhibition of target gene expression. Thus, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified further optimization can be achieved by systematically either adding or removing nucleotides to generate longer or shorter sequences and testing those and sequences generated by walking a window of the longer or shorter size up or down the target RNA from that point. Again, coupling this approach to generating new candidate targets with testing for effectiveness of siRNAs based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An iRNA as described herein can contain one or more mismatches to the target sequence. In one embodiment, an siRNA as described herein contains no more than 3 mismatches. If the antisense strand of the siRNA contains mismatches to a target sequence, it is preferable that the area of mismatch not be located in the center of the region of complementarity. If the antisense strand of the siRNA contains mismatches to the target sequence, it is preferable that the mismatch be restricted to be within the last 5 nucleotides from either the 5′ or 3′ end of the region of complementarity. For example, for a 23 nucleotide siRNA agent RNA strand which is complementary to a region of a target gene, the RNA strand generally does not contain any mismatch within the central 13 nucleotides. The methods described herein or methods known in the art can be used to determine whether an siRNA containing a mismatch to a target sequence is effective in inhibiting the expression of a target gene. Consideration of the efficacy of siRNAs with mismatches in inhibiting expression of a target gene is important, especially if the particular region of complementarity in a target gene is known to have polymorphic sequence variation within the population.

In one embodiment, at least one end of a dsRNA has a single-stranded nucleotide overhang of 1 to 4, generally 1 or 2 nucleotides. dsRNAs having at least one nucleotide overhang have unexpectedly superior inhibitory properties relative to their blunt-ended counterparts. In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S.L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in this invention include, but are not limited to RNAs containing modified backbones or no natural internucleoside linkages RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6, 239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and US Pat RE39464, each of which is herein incorporated by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in siRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs may also contain one or more substituted sugar moieties. The siRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)_{·n}OCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an siRNA, and other substituents having similar properties. In some embodiments, the modification includes a 2′-methoxyethoxy (2′-O—OH₂CH₂OCH₃, also known as 2′-O-(2-methoxyethyl) or 2′-MOE) (Martin et al., Helv. Chin. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F) Similar modifications may also be made at other positions on the RNA of an siRNA, particularly the 3′ position of the sugar on the 3′ terminal nucleotide or in 2′-5′ linked dsRNAs and the 5′ position of 5′ terminal nucleotide. siRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An siRNA may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of a siRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193).

Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-0-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

iRNA Motifs

In one embodiment, the sense strand sequence may be represented by formula (I):

5′ nₚ-Nₐ-(X X X)ᵢ-N_{b}-Y Y Y-N_{b}-(Z Z Z)ⱼ-Nₐ-n_{q} 3′ (I)

wherein:

i and j are each independently 0 or 1;

p and q are each independently 0-6;

each Nₐ independently represents an oligonucleotide sequence comprising 0-25 modified nucleotides, each sequence comprising at least two differently modified nucleotides;

each N_{b} independently represents an oligonucleotide sequence comprising 0-10 modified nucleotides;

each nₚ and n_{q} independently represent an overhang nucleotide;

wherein Nb and Y do not have the same modification; and

XXX, YYY and ZZZ each independently represent one motif of three identical modifications on three consecutive nucleotides. Preferably YYY is all 2′-F modified nucleotides.

In one embodiment, the Nₐ and/or N_{b} comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8; 7, 8, 9; 8, 9, 10; 9, 10, 11; 10, 11,12 or 11, 12, 13) of —the sense strand, the count starting from the 1^{st} nucleotide, from the 5′-end; or optionally, the count starting at the 1^{st} paired nucleotide within the duplex region, from the 5′-end.

In one embodiment, i is 1 and j is 0, or i is 0 and j is 1, or both i and j are 1. The sense strand can therefore be represented by the following formulas:

5′ nₚ-Nₐ-YYY-N_{b}-ZZZ-Nₐ-n_{q} 3′ (Ib);

5′ nₚ-Nₐ-XXX-N_{b}-YYY-Nₐ-n_{q} 3′ (Ic); or

5′ nₚ-Nₐ-XXX-N_{b}-YYY-N_{b}-ZZZ-Nₐ-n_{q} 3′ (Id).

When the sense strand is represented by formula (Ib), N_{b} represents an oligonucleotide sequence comprising 0-10, 0-7, 0-5, 0-4, 0-2 or 0 modified nucleotides. Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-5, 0-4, 0-2 or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2 or 0 modified nucleotides. Preferably, N_{b} is 0, 1, 2, 3, 4, 5 or 6. Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

5′ nₚ-Nₐ-YYY-Nₐ-n_{q} 3′ (Ia).

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

5′ n_{q′}-Nₐ′-(Z′Z′Z′)ₖ-N_{b}′-Y′Y′Y′-N_{b}′-(X′X′X′)ₗ-N′ₐ-nₚ′3′ (II)

wherein:

k and l are each independently 0 or 1;

p′ and q′ are each independently 0-6;

each Nₐ′ independently represents an oligonucleotide sequence comprising 0-25 modified nucleotides, each sequence comprising at least two differently modified nucleotides;

each N_{b}′ independently represents an oligonucleotide sequence comprising 0-10 modified nucleotides;

each nₚ′ and n_{q}′ independently represent an overhang nucleotide;

wherein N_{b}′ and Y′ do not have the same modification; and

X′X′X′, Y′Y′Y′ and Z′Z′Z′ each independently represent one motif of three identical modifications on three consecutive nucleotides.

In one embodiment, the Nₐ′ and/or N_{b}′ comprise modifications of alternating pattern.

The Y′Y′Y′ motif occurs at or near the cleavage site of the antisense strand. For example, when the RNAi agent has a duplex region of 17-23 nucleotidein length, the Y′Y′Y′ motif can occur at positions 9, 10, 11;10, 11, 12; 11, 12, 13; 12, 13, 14 ; or 13, 14, 15 of the antisense strand, with the count starting from the 1^{st} nucleotide, from the 5′-end; or optionally, the count starting at the 1^{st} paired nucleotide within the duplex region, from the 5′-end. Preferably, the Y′Y′Y′ motif occurs at positions 11, 12, 13.

In one embodiment, Y′Y′Y′ motif is all 2′-OMe modified nucleotides.

In one embodiment, k is 1 and 1 is 0, or k is 0 and 1 is 1, or both k and l are 1.

The antisense strand can therefore be represented by the following formulas:

5′ n_{q}′-Nₐ′-Z′Z′Z′-N_{b}′-Y′Y′Y′-Nₐ′-nₚ′3′ (IIb);

5′ n_{q}′-Nₐ′-Y′Y′Y′-N_{b}′-X′X′X′-nₚ′3′ (IIc); or

5′ n_{q}′-Nₐ′-X′X′X′-Nₐ′-nₚ′3′ (IId).

When the antisense strand is represented by formula (IIb), N_{b}′ represents an oligonucleotide sequence comprising 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-5, 0-4, 0-2 or 0 modified nucleotides. Each Nₐ′ independently represents an oligonucleotide sequence comprising 2-20, 2-15, or 2-10 modified nucleotides. Preferably, N_{b} is 0, 1, 2, 3, 4, 5 or 6.

In other embodiments, k is 0 and l is 0 and the antisense strand may be represented by the formula:

5′ nₚ′-Nₐ′-Y′Y′Y′-N_{a′}-n_{q′}3′ (Ia).

When the antisense strand is represented as formula (IIa), each Nₐ′ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, HNA, CeNA, 2′ -methoxyethyl, 2′-O-methyl, 2′-O-allyl, 2′-C- allyl, 2′-hydroxyl, or 2′-fluoro. For example, each nucleotide of the sense strand and antisense strand is independently modified with 2′-O-methyl or 2′-fluoro. Each X, Y, Z, X′, Y′ and Z′, in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nucleotides, the count starting from the 1^{st} nucleotide from the 5′-end, or optionally, the count starting at the 1^{st} paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y′ motif occurring at positions 11, 12, 13 of the strand, the count starting from the P^{t} nucleotide from the 5′-end, or optionally, the count starting at the 1^{st} paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with a antisense strand being represented by any one of formulas (IIa), (IIb), (IIc), and (IId), respectively.

Accordingly, the RNAi agents for use in the methods of the invention may comprise a sense strand and an antisense strand, each strand having 14 to 30 nucleotides, the RNAi duplex represented by formula (III):

sense: 5′ nₚ -Nₐ-(X X X)ᵢ-N_{b}-Y Y Y-N_{b}-(Z Z Z)ⱼ-Nₐ-n_{q} 3′

antisense: 3′ nₚ′-Nₐ′-(X′X′X′)ₖ-N_{b}′-Y′Y′Y′-N_{b}′-(Z′Z′Z′)ᵢ-Nₐ′-n_{q}′5′ (III)

wherein:

j, k, and l are each independently 0 or 1;

p, p′, q, and q′ are each independently 0-6;

each Nₐ and Nₐ′ independently represents an oligonucleotide sequence comprising 0-25 modified nucleotides, each sequence comprising at least two differently modified nucleotides;

each N_{b} and N_{b}′ independently represents an oligonucleotide sequence comprising 0-10 modified nucleotides;

wherein

each nₚ′, nₚ, n_{q}′, and n_{q}, each of which may or may not be present, independently represents an overhang nucleotide; and

XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and l is 0; or k is 1 and 1 is 0; k is 0 and l is 1; or both k and l are 0; or both k and l are 1.

Exemplary combinations of the sense strand and antisense strand forming a RNAi duplex include the formulas below:

5′ nₚ -Nₐ-Y Y Y-Nₐ-n_{q} 3′

3′ nₚ′-Nₐ′-Y′Y′Y′ -Nₐ′n_{q}′5′ (IIIa)

5′ nₚ-Y Y Y-N_{b}-Z Z Z-Nₐ-n_{q} 3′

3′ nₚ′-Nₐ′-Y′Y′Y′-N_{b}′-Z′Z′Z′-Nₐ′n_{q}′5′ (IIIb)

5′ nₚ-Nₐ-X X X-N_{b}-Y Y Y-Nₐ-n_{q} 3′

3′ nₚ′-Nₐ′-X′X′X′-N_{b}′-Y′Y′Y′-Nₐ′-n_{q}′5′ (IIIc)

5′ nₚ-XXX -N_{b}-Y Y Y-N_{b}-Z Z Z-Nₐ-n_{q} 3′

3′ nₚ′-Nₐ′-X′X′X′-N_{b}′-Y′Y′Y′-N_{b}′-Z′Z′Z′-Nₐ-n_{q}′5′ (IIId)

When the RNAi agent is represented by formula (IIIa), each Nₐ independently represents an oligonucleotide sequence comprising 2-20, 2-15, or 2-10 modified nucleotides.

When the RNAi agent is represented by formula (IIIb), each N_{b} independently represents an oligonucleotide sequence comprising 1-10, 1-7, 1-5 or 1-4 modified nucleotides. Each Nₐ independently represents an oligonucleotide sequence comprising 2-20, 2-15, or 2-10 modified nucleotides.

When the RNAi agent is represented as formula (IIIc), each N_{b}, N_{b}′ independently represents an oligonucleotide sequence comprising 0-10, 0-7, 0-5, 0-4, 0-2 or 0 modified nucleotides. Each Nₐ independently represents an oligonucleotide sequence comprising 2-20, 2-15, or 2-10 modified nucleotides.

When the RNAi agent is represented as formula (IIId), each N_{b}, N_{b}′ independently represents an oligonucleotide sequence comprising 0-10, 0-7, 0-5, 0-4, 0-2 or 0 modified nucleotides. Each Nₐ, Nₐ′ independently represents an oligonucleotide sequence comprising 2-20, 2-15, or 2-10 modified nucleotides. Each of Nₐ, Nₐ′, N_{b} and N_{b}′ independently comprises modifications of alternating pattern.

Each of X, Y and Z in formulas (III), (IIIa), (IIIb), (IIIc), and (IIId) may be the same or different from each other.

When the RNAi agent is represented by formula (III), (IIIa), (IIIb), (IIIc), and (IIId), at least one of the Y nucleotides may form a base pair with one of the Y′ nucleotides. Alternatively, at least two of the Y nucleotides form base pairs with the corresponding Y′ nucleotides; or all three of the Y nucleotides all form base pairs with the corresponding Y′ nucleotides.

When the RNAi agent is represented by formula (IIIb) or (IIId), at least one of the Z nucleotides may form a base pair with one of the Z′ nucleotides. Alternatively, at least two of the Z nucleotides form base pairs with the corresponding Z′ nucleotides; or all three of the Z nucleotides all form base pairs with the corresponding Z′ nucleotides.

When the RNAi agent is represented as formula (IIIc) or (IIId), at least one of the X nucleotides may form a base pair with one of the X′ nucleotides. Alternatively, at least two of the X nucleotides form base pairs with the corresponding X′ nucleotides; or all three of the X nucleotides all form base pairs with the corresponding X′ nucleotides.

In one embodiment, the modification on the Y nucleotide is different than the modification on the Y′ nucleotide, the modification on the Z nucleotide is different than the modification on the Z′ nucleotide, and/or the modification on the X nucleotide is different than the modification on the X′ nucleotide.

In one embodiment, when the RNAi agent is represented by formula (IIId), the Nₐ modifications are 2′-O-methyl or 2′-fluoro modifications. In another embodiment, when the RNAi agent is represented by formula (IIId), the Nₐ modifications are 2′-O-methyl or 2′-fluoro modifications and nₚ′>0 and at least one nₚ′ is linked to a neighboring nucleotide a via phosphorothioate linkage In yet another embodiment, when the RNAi agent is represented by formula (IIId), the Nₐ modifications are 2′-O-methyl or 2′-fluoro modifications , nₚ′>0 and at least one nₚ′ is linked to a neighboring nucleotide via phosphorothioate linkage, and the sense strand is conjugated to one or more GalNAc derivatives attached through a bivalent or trivalent branched linker In another embodiment, when the RNAi agent is represented by formula (IIId), the Nₐ modifications are 2′-O-methyl or 2′-fluoro modifications , nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, when the RNAi agent is represented by formula (IIIa), the Nₐ modifications are 2′-O-methyl or 2′-fluoro modifications , nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In one embodiment, the RNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes ca target the same gene or two different genes; or each of the duplexes ca target same gene at two different target sites.

In one embodiment, the RNAi agent is a multimer containing three, four, five, six or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes ca target the same gene or two different genes; or each of the duplexes ca target same gene at two different target sites.

In one embodiment, two RNAi agents represented by formula (III), (IIIa), (IIIb), (IIIc), and (IIId) are linked to each other at the 5′ end, and one or both of the 3′ ends and are optionally conjugated to to a ligand. Each of the agents ca target the same gene or two different genes; or each of the agents ca target same gene at two different target sites.

Nucleic Acid (e.g., iRNA) Conjugates

The nuclec acid (e.g., iRNA) agents disclosed herein can be in the form of conjugates. The conjugate may be attached at any suitable location in the siRNA molecule, e.g., at the 3′ end or the 5′ end of the sense or the antisense strand. The conjugates are optionally attached via a linker

In some embodiments, an iRNA agent described herein is chemically linked to one or more ligands, moieties or conjugates, which may confer functionality, e.g., by affecting (e.g., enhancing) the activity, cellular distribution or cellular uptake of the siRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In one embodiment, a ligand alters the distribution, targeting or lifetime of an siRNA agent into which it is incorporated. In some embodiments, a ligand provides an enhanced affinity for a selected target, e.g, molecule, cell or cell type, compartment, e.g., a cellular or organ compartment, tissue, organ or region of the body, as, e.g., compared to a species absent such a ligand. Typical ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an a helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic.

In some embodiments, the ligand is a GalNAc ligand that comprises one or more N-acetylgalactosamine (GalNAc) derivatives. Additional description of GalNAc ligands is provided in the section titled Carbohydrate Conjugates.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be, for example, a lipopolysaccharide, an activator of p38 MAP kinase, or an activator of NF-ηB.

The ligand can be a substance, e.g., a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an siRNA as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated oligonucleotides of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### Lipid Conjugates

In one embodiment, the ligand is a lipid or lipid-based molecule. Such a lipid or lipid-based molecule can typically bind a serum protein, such as human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control (e.g., inhibit) the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In one embodiment, the lipid based ligand binds HSA. For example, the ligand can bind HSA with a sufficient affinity such that distribution of the conjugate to a non-kidney tissue is enhanced. However, the affinity is typically not so strong that the HSA-ligand binding cannot be reversed.

In another embodiment, the lipid based ligand binds HSA weakly or not at all, such that distribution of the conjugate to the kidney is enhanced. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

### Cell Permeation Agents

In another aspect, the ligand is a cell-permeation agent, such as a helical cell-permeation agent. In one embodiment, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is typically an a-helical agent, and can have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 1). An RFGF analogue (e.g., amino acid sequence AALLPVLLAAP (SEQ ID NO: 2)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 3)) and the Drosophila Antennapedia protein (RQIKIWFQNRRMKWKK (SEQ ID NO: 4)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Typically, the peptide or peptidomimetic tethered to a dsRNA agent via an incorporated monomer unit is a cell targeting peptide such as an arginine-glycine-aspartic acid (RGD)-peptide, or RGD mimic A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimemtics may include D-amino acids, as well as synthetic RGD mimics In addition to RGD, one can use other moieties that target the integrin ligand. Preferred conjugates of this ligand target PECAM-1 or VEGF.

An RGD peptide moiety can be used to target a particular cell type, e.g., a tumor cell, such as an endothelial tumor cell or a breast cancer tumor cell (Zitzmann et al., Cancer Res., 62:5139-43, 2002). An RGD peptide can facilitate targeting of an dsRNA agent to tumors of a variety of other tissues, including the lung, kidney, spleen, or liver (Aoki et al., Cancer Gene Therapy 8: 783-787, 2001). Typically, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver a iRNA agent to a tumor cell expressing avB3 (Haubner et al., Jour. Nucl. Med., 42:326-336, 2001).

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### Carbohydrate Conjugates

In some embodiments of the compositions and methods of the invention, an iRNA oligonucleotide further comprises a carbohydrate. The carbohydrate conjugated iRNA are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one embodiment, a carbohydrate conjugate comprises a monosaccharide. In one embodiment, the monosaccharide is an N-acetylgalactosamine (GalNAc). GalNAc conjugates are described, for example, in U.S. Pat. No. 8,106,022, the entire content of which is hereby incorporated herein by reference. In some embodiments, the GalNAc conjugate serves as a ligand that targets the siRNA to particular cells. In some embodiments, the GalNAc conjugate targets the siRNA to liver cells, e.g., by serving as a ligand for the asialoglycoprotein receptor of liver cells (e.g., hepatocytes).

In some embodiments, the carbohydrate conjugate comprises one or more GalNAc derivatives. The GalNAc derivatives may be attached via a linker, e.g., a bivalent or trivalent branched linker In some embodiments the GalNAc conjugate is conjugated to the 3′ end of the sense strand. In some embodiments, the GalNAc conjugate is conjugated to the iRNA agent (e.g., to the 3′ end of the sense strand) via a linker, e.g., a linker as described herein.

In some embodiments, the GalNAc conjugate is

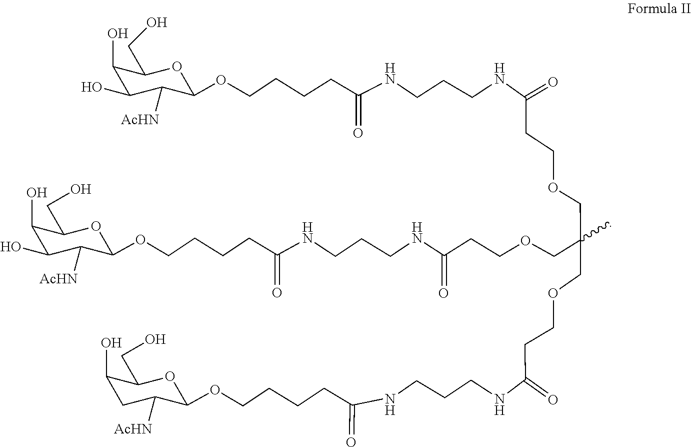 [SMILES: CC(=O)NC1CC(O)C(CO)OC1OCCCCC(=O)NCCCNC(=O)CCOCC(C)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, the RNAi agent is conjugated to L96 as defined in Table 1 and shown below

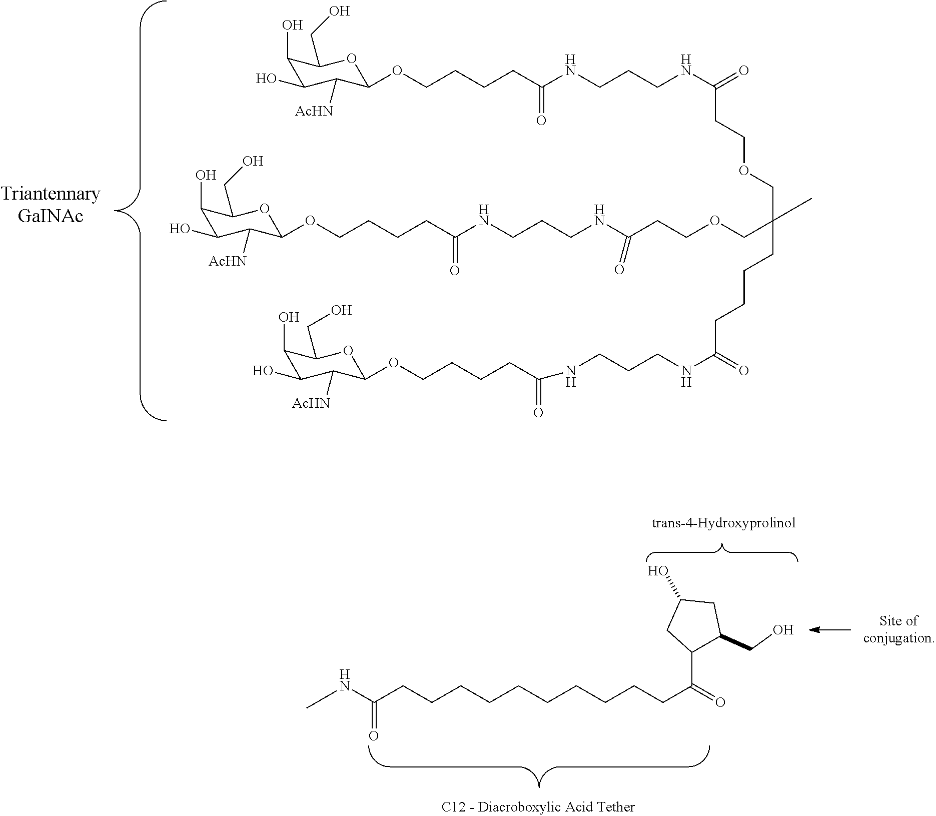

In some embodiments, the carbohydrate conjugate further comprises one or more additional ligands as described above, such as, but not limited to, a PK modulator and/or a cell permeation peptide.

In one embodiment, an siRNA of the invention is conjugated to a carbohydrate through a linker.

### Linkers

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-16, or 8-16 atoms.

In one embodiment, a dsRNA of the invention is conjugated to a bivalent or trivalent branched linker selected from the group of structures shown in any of formula (XXXI)-(XXXIV):

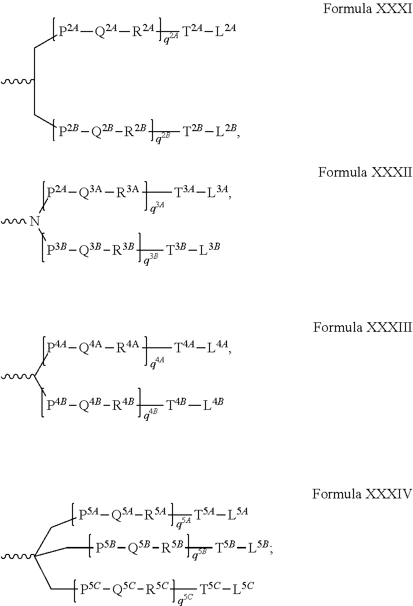

wherein:

q2A, q2B, q3A, q3B, q4A, q4B, q5A, q5B and q5C represent independently for each occurrence 0-20 and wherein the repeating unit can be the same or different;

p^{2A}, p^{2B}, p^{3A}, p^{3B}, p^{4A}, p^{4B}, p^{5A}, p^{5B}, p^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;

Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherin one or more methylenes can be interrupted or terminated by one or more of O, S, S(O), SO₂, N(R^{N}), C(R′)═C(R″), C≡C or C(O); R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

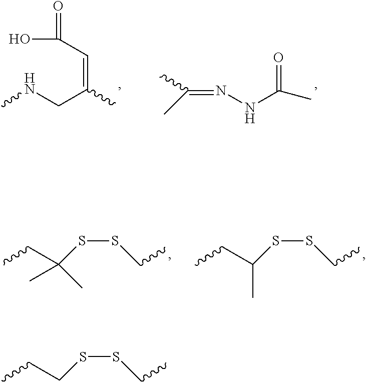

or heterocyclyl;

L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; andRa is H or amino acid side chain. Trivalent conjugating GalNAc derivatives are particularly useful for use with RNAi agents for inhibiting the expression of a target gene, such as those of formula (XXXV):

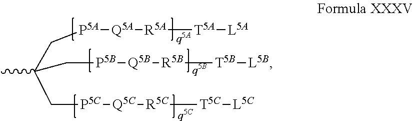

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

### Redox Cleavable Linking Groups

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents known in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Phosphate-Based Cleavable Linking Groups

In another embodiment, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### Acid Cleavable Linking Groups

In another embodiment, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Ester-Based Cleavable Linking Groups

In another embodiment, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### Peptide-Based Cleavable Linking Groups

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which is herein incorporated by reference.

It is not necessary for all positions in a given compound to be uniformly modified, and in fact more than one of the aforementioned modifications may be incorporated in a single compound or even at a single nucleoside within an iRNA. The present invention also includes iRNA compounds that are chimeric compounds.

“Chimeric” nucleic acid (e.g., iRNA) compounds, or “chimeras,” in the context of the present invention, are nucleic acid (e.g., iRNA) compounds, e.g., dsRNAs, that contain two or more chemically distinct regions, each made up of at least one monomer unit, i.e., a nucleotide in the case of a dsRNA compound. These compounds typically contain at least one region wherein the RNA is modified so as to confer upon the iRNA increased resistance to nuclease degradation, increased cellular uptake, and/or increased binding affinity for the target nucleic acid. An additional region of the compounds may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an siRNA can be modified by a non-ligand group. A number of non-ligand molecules have been conjugated to siRNAs in order to enhance the activity, cellular distribution or cellular uptake of the siRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of an RNAs bearing an aminolinker at one or more positions of the sequence. The amino group is then reacted with the molecule being conjugated using appropriate coupling or activating reagents. The conjugation reaction may be performed either with the RNA still bound to the solid support or following cleavage of the RNA, in solution phase. Purification of the RNA conjugate by HPLC typically affords the pure conjugate.

### Target Genes and Methods for Treating Diseases Related to Expression of a Target Gene

The assays and methods described herein can be used to detect antibodies against a nucleic acid molecule (e.g., an RNA molecule) that inhibits target gene expression. In certain embodiments, the target genes is chosen from: Factor VII, Eg5, PCSK9, TPX2, apoB, SAA, TTR, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, HAMP, Activated Protein C gene, Cyclin D gene, VEGF gene, antithrombin 3 gene, aminolevulinate synthase 1 gene, alpha-1-antitrypsin gene, tmprss6 gene, apoal gene, apoc3 gene, bclla gene, klf gene, angpt13 gene, plk gene, PKN3 gene, HBV, HCV, p53 gene, angiopoietin gene, or angiopoietin-like 3 gene. In certain embodiments, the target is chosen from: Eg5, PCSK9, TTR, HAMP, VEGF gene, antithrombin 3 gene, aminolevulinate synthase 1 gene, alpha-1-antitrypsin gene, tmprss6 gene, complement C5 gene, or complement C3 gene.

### TTR Target Gene

In one embodiment, the target gene is a TTR gene. Nucleic acid (e.g., RNA) molecules capable of reducing expression of a TTR gene (e.g., to reduce TTR amyloid deposition, or treating a TTR-mediated amyloidosis (ATTR)) are described in, e.g., WO 2011/056883, the contents of which are specifically incorporated by reference herein. In certain embodiments, the RNA molecule comprises an antisense strand comprising, or consisting of, 10, 15, 20, 25 or more contiguous nucleotides complementary to the transthyretin mRNA (e.g., wild type or mutant TTR mRNA e.g., V30M mutant TTR). In certain embodiments, the RNA molecule comprises an antisense strand comprising, or consisting of, 10, 15, 20, 25 or more contiguous nucleotides of an antisense oligonucleotide sequence disclosed in, e.g., WO 2011/056883, e.g., SEQ ID NOs: 170, 730, or 1010. In certain embodiments, the RNA molecule comprises an antisense strand comprising, or consisting of, 10, 15, 20, 25 or more contiguous nucleotides of an antisense oligonucleotide sequence disclosed in, e.g., WO 2011/056883, e.g., SEQ ID NOs: 170, 730, or 1010; and a sense strand disclosed in, e.g., WO 2011/056883, e.g., SEQ ID NOs: 169, 729, or 1009.

### PCSK9 Target Gene

In one embodiment, the target gene is a PCSK9 gene. Nucleic acid (e.g., RNA) molecules capable of reducing expression of a PCSK9 gene (e.g., to treat a PCSK9-related disorder, e.g., lowering serum cholesterol) are described, e.g., in WO 2012/05869, WO 2011/005861, WO 2011/028938, WO 2010/148013, WO 2009/134487 and WO 2007/134161, the contents of which are specifically incorporated by reference herein. In certain embodiments, the RNA molecule comprises an antisense strand comprising 10, 15, 20, 25 or more contiguous nucleotides complementary to the PCSK9 mRNA (e.g., wild type or mutant PCSK9 mRNA). In certain embodiments, the RNA molecule comprises an antisense strand comprising, or consisting of, 10, 15, 20, 25 or more contiguous nucleotides of an antisense oligonucleotide sequence disclosed, e.g., in WO 2012/05869, WO 2011/005861, WO 2011/028938, WO 2010/148013, WO 2009/134487 and WO 2007/134161. In certain embodiments, the RNA molecule comprises an antisense strand comprising, or consisting of, 10, 15, 20, 25 or more contiguous nucleotides of an antisense oligonucleotide sequence disclosed, e.g., in WO 2012/05869, WO 2011/005861, WO 2011/028938, WO 2010/148013, WO 2009/134487 and WO 2007/134161; and a sense strand disclosed, e.g., in WO 2012/05869, WO 2011/005861, WO 2011/028938, WO 2010/148013, WO 2009/134487 and WO 2007/134161.

### Eg5 Target Gene

In one embodiment, the target gene is an Eg5 gene. Nucleic acid (e.g., RNA) molecules capable of reducing expression of an Eg5 gene (e.g., to treat an Eg5-related disorder) are described, e.g., in WO 2011/034798, WO 2010/105209, WO 2009/111658, and WO 2007/115168, the contents of which are specifically incorporated by reference herein. In certain embodiments, the RNA molecule comprises an antisense strand comprising 10, 15, 20, 25 or more contiguous nucleotides complementary to the Eg5 mRNA (e.g., wild type or mutant Eg5 mRNA). In certain embodiments, the RNA molecule comprises an antisense strand comprising, or consisting of, 10, 15, 20, 25 or more contiguous nucleotides of an antisense oligonucleotide sequence disclosed, e.g., in WO 2011/034798, WO 2010/105209, WO 2009/111658, and WO 2007/115168. In certain embodiments, the RNA molecule comprises an antisense strand comprising, or consisting of, 10, 15, 20, 25 or more contiguous nucleotides of an antisense oligonucleotide sequence disclosed, e.g., in WO 2011/034798, WO 2010/105209, WO 2009/111658, and WO 2007/115168; and a sense strand disclosed, e.g., in WO 2011/034798, WO 2010/105209, WO 2009/111658, and WO 2007/115168.

### VEGF Target Gene

In one embodiment, the target gene is a VEGF gene. Nucleic acid (e.g., RNA) molecules capable of reducing expression of a VEGF gene (e.g., to treat a VEGF- related disorder) are described, e.g., in WO 2011/034798, WO 2010/105209, WO 2009/111658, and WO 2005/089224, the contents of which are specifically incorporated by reference herein. In certain embodiments, the RNA molecule comprises an antisense strand comprising 10, 15, 20, 25 or more contiguous nucleotides complementary to the VEGF mRNA (e.g., wild type or mutant VEGF mRNA). In certain embodiments, the RNA molecule comprises an antisense strand comprising, or consisting of, 10, 15, 20, 25 or more contiguous nucleotides of an antisense oligonucleotide sequence disclosed, e.g., in WO 2011/034798, WO 2010/105209, WO 2009/111658, and WO 2005/089224. In certain embodiments, the RNA molecule comprises an antisense strand comprising, or consisting of, 10, 15, 20, 25 or more contiguous nucleotides of an antisense oligonucleotide sequence disclosed, e.g., in WO 2011/034798, WO 2010/105209, WO 2009/111658, and WO 2005/089224; and a sense strand disclosed, e.g., in WO 2011/034798, WO 2010/105209, WO 2009/111658, and WO 2005/089224.

### HAMP Target Gene

In one embodiment, the target gene is a Hepcidin Antimicrobial Peptide (HAMP) gene. Nucleic acid (e.g., RNA) molecules capable of reducing expression of a HAMP gene (e.g., to treat a HAMP—related disorder, e.g., a microbial infection) are described, e.g., in WO 2008/036933 and WO 2012/177921, the contents of which are specifically incorporated by reference herein. In certain embodiments, the RNA molecule comprises an antisense strand comprising 10, 15, 20, 25 or more contiguous nucleotides complementary to the HAMP mRNA (e.g., wild type or mutant HAMP mRNA). In certain embodiments, the RNA molecule comprises an antisense strand comprising, or consisting of, 10, 15, 20, 25 or more contiguous nucleotides of an antisense oligonucleotide sequence disclosed, e.g., in WO 2008/036933 and WO 2012/177921. In certain embodiments, the RNA molecule comprises an antisense strand comprising, or consisting of, 10, 15, 20, 25 or more contiguous nucleotides of an antisense oligonucleotide sequence disclosed, e.g., in WO 2008/036933 and WO 2012/177921; and a sense strand disclosed, e.g., in WO 2008/036933 and WO 2012/177921.

### TMPRSS6 Target Gene

In one embodiment, the target gene is a TMPRSS6 gene. Nucleic acid (e.g., RNA) molecules capable of reducing expression of a TMPRSS6 gene (e.g., to treat a TMPRSS6 -related disorder) are described, e.g., in WO 2012/135246, the contents of which are specifically incorporated by reference herein. In certain embodiments, the RNA molecule comprises an antisense strand comprising 10, 15, 20, 25 or more contiguous nucleotides complementary to the TMPRSS6 mRNA (e.g., wild type or mutant TMPRSS6 mRNA). In certain embodiments, the RNA molecule comprises an antisense strand comprising, or consisting of, 10, 15, 20, 25 or more contiguous nucleotides of an antisense oligonucleotide sequence disclosed, e.g., in WO 2012/135246. In certain embodiments, the RNA molecule comprises an antisense strand comprising, or consisting of, 10, 15, 20, 25 or more contiguous nucleotides of an antisense oligonucleotide sequence disclosed, e.g., in WO 2012/135246; and a sense strand disclosed, e.g., in WO 2012/135246.

### 5′-Aminolevulinic Acid Synthase 1 (ALAS1) Gene

In one embodiment, the target gene is an ALAS1 gene. Nucleic acid (e.g., RNA) molecules capable of reducing expression of an ALAS1 gene (e.g., to treat an ALAS1-related disorder, e.g. a pathological processes involving porphyrins or defects in the porphyrin pathway, such as, for example, porphyrias) are described, e.g., in U.S. Ser. No. 13/835,613, filed on Mar. 15, 2013, the contents of which are specifically incorporated by reference herein. In certain embodiments, the RNA molecule comprises an antisense strand comprising 10, 15, 20, 25 or more contiguous nucleotides complementary to the ALAS1 mRNA (e.g., wild type or mutant ALAS1 mRNA). In certain embodiments, the RNA molecule comprises an antisense strand comprising, or consisting of, 10, 15, 20, 25 or more contiguous nucleotides of an antisense oligonucleotide sequence disclosed, e.g., in WO2013/155204. In certain embodiments, the RNA molecule comprises an antisense strand comprising, or consisting of, 10, 15, 20, 25 or more contiguous nucleotides of an antisense oligonucleotide sequence disclosed, e.g., in WO2013/155204; and a sense strand disclosed, e.g., in WO2013/155204.

### Complement Component 3 (C3) Gene

In one embodiment, the target gene is a Complement component 3 (C3) gene. Nucleic acid (e.g., RNA) molecules capable of reducing expression of a C3 gene (e.g., to treat a C3 -related disorder. C3 plays a central role in the complement system and contributes to innate immunity. In humans it is encoded on chromosome 19 by a gene called C3. In certain embodiments, the RNA molecule comprises an antisense strand comprising 10, 15, 20, 25 or more contiguous nucleotides complementary to the C5 mRNA (e.g., wild type or mutant C3 mRNA).

### Complement Component 5 (C5) Gene

In one embodiment, the target gene is a Complement component 5 (C5) gene. Nucleic acid (e.g., RNA) molecules capable of reducing expression of a C5 gene (e.g., to treat a C5 -related disorder, e.g. a pathological processes involving inflammatory and cell killing processes. This protein is composed of alpha and beta polypeptide chains that are linked by a disulfide bridge. An activation peptide, C5a, which is an anaphylatoxin that possesses potent spasmogenic and chemotactic activity, is derived from the alpha polypeptide via cleavage with a convertase. In certain embodiments, the RNA molecule comprises an antisense strand comprising 10, 15, 20, 25 or more contiguous nucleotides complementary to the C5 mRNA (e.g., wild type or mutant C5 mRNA).

### Immobilization of Nucleic Acid Molecules

The nucleic acid molecules (e.g., an oligonucleotide molecule (e.g., a double-stranded oligonucleotide), or an RNA molecule, e.g., a double-stranded RNA (dsRNA)), used in the methods and assays described herein can be immobilized to a solid surface, e.g., the surface of a plate (e.g., microwell plate) using techniques and reagents known in the art.

In one embodiment, the nucleic acid molecule is covalently immobilized to the plate. For example, the nucleic acid molecule can be phosphorylated at the 5′-end, e.g., the 5′-end of a sense or an antisense strand, or both. The phosphorylated (e.g., 5′ phosphorylated) nucleic acid molecule can be immobilized to a surface coated via a reactive group, e.g., a plate coated with a reactive group chosen from an amine (e.g., secondary amino) group or a sulfhydryl group. In some embodiments, the phosphate group of the nucleic acid (e.g., RNA) molecule forms a covalent bond (e.g., a phosphoramidate bond) with the reactive group (e.g., the secondary amino group) present on the surface of the plate. Exemplary methods of covalent immobilization of DNA onto polystyrene microwells for hybridization are described in Rasmussen et al. Analytical Biochemistry 198, 138-142 (1991), incorporated herein by reference.

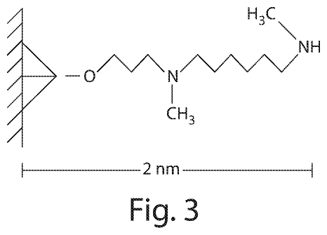

The reactive group may optionally comprise a linker. The linker can also include a spacer arm that is covalently grated to the plate surface. The reactive group can be positioned at the end of the spacer arm as depicted in FIG. 3. The density of the reactive group on the plate may vary. For example, the density can be between about 10¹⁰/cm² and about 10¹⁶/cm², e.g., between about 10¹²/cm² and about 10¹⁴/cm², e.g., about 10¹²/cm², about 10¹³/cm², about 10¹⁴/cm², about 10¹⁵/cm², or about 10¹⁶/cm².

In another embodiment, the nucleic acid molecule is immobilized to the solid support via non-covalent (e.g., affinity) interaction. For example, the plate can be coated with an affinity agent that interacts with a partner moiety coupled to the nucleic acid molecule. Exemplary affinity agents include a protein or ligand of a protein-ligand pair, e.g., biotin-streptavidin. In one embodiment, the solid surface, e.g., plate, is be coated with streptavidin such that a biotinylated RNA molecule can be immobilized to the plate through the streptavidin-biotin affinity interaction.

In yet another embodiment, the nucleic acid molecule is immobilized to the solid support via an antigen-antibody interaction. For example, the plate can be coated with an antibody to the nucleic acid molecule such that the double stranded oligonucleotides or nucleic acid molecule can be immobilized to the plate through the antigen-antibody interaction.

Various types of solid supports, e.g., plates, can be coated with the nucleic acid molecule or non-covalent partners. Suitable solid phase supports include any support capable of binding a nucleic acid, a protein or an antibody. Exemplary supports include glass, polystyrene, polypropylene, polyethylene, dextran, nylon, amylases, natural and modified celluloses, polyacrylamides, gabbros, and magnetite.

In one embodiment, the solid support, e.g., plate, is a polystyrene plate. The plate (e.g., polystyrene plate) can be grafted with a reactive group, e.g., an amine (e.g., a secondary amino) group or a sulfhydryl group. For example, the reactive group can be positioned at the end of a spacer arm that is covalently grafted to the surface of the plate. In one embodiment, the plate is coated with a secondary amino group (e.g., a CovaLink™ NH plate (Nalge-Nunc, Product No. 478042)). In another embodiment, the plate is a maleimide activated plate (e.g., Pierce Maleimide Activated Plates Product Nos. 15150, 15152 and 15153). In yet another embodiment, the plate is coated with streptavidin (e.g., Pierce Streptavidin Coated High Sensitivity Plates, Product Nos. 15520 and 15525; or Nunc Immobilizer, Solid plate 96-well, Flat-bottom, Streptavidin covalently coated, 400 μL, Thermo Scientific Nos. 436014 and 436015).

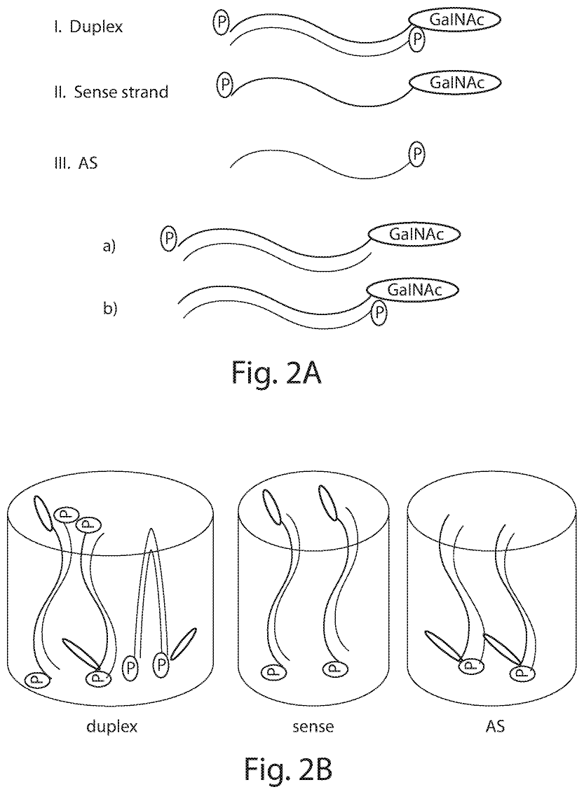

The nucleic acid molecules (e.g., double-stranded oligonucleotide or RNA molecule, e.g., dsRNA) immobilized to the solid support, e.g., plate, can exhibit various orientations. In some embodiments, the nucleic acid molecule comprises at least two strands (e.g., a sense strand and an antisense strand). In one embodiment, the sense strand is immobilized to the plate. In another embodiment, the antisense strand is immobilized to the plate. In yet another embodiment, both the sense strand and the antisense strand are immobilized to the plate. In some embodiments, the nucleic acid molecule is immobilized to the plate at one end of the molecule or strand (e.g., 5′ end or 3′ end). For example, the nucleic acid molecule can be immobilized to the plate at the 5′ end of the sense strand, 5′ end of the antisense strand, or both. In other embodiments, the nucleic acid molecule is immobilized to the plate at both ends of the molecule or strand (e.g., 5′ end and 3′ end). Exemplary orientations of the nucleic acid molecules (e.g., double-stranded oligonucleotide or RNA molecule, e.g., dsRNA) are depicted in FIG. 2B.

The nucleic acid molecules (e.g., double-stranded oligonucleotide or RNA molecule, e.g., dsRNA) to be immobilized to the solid support, e.g., plate, can contain a phosphate group at the end of the molecule. In one embodiment, the nucleic acid molecule (e.g., double-stranded oligonucleotide or RNA molecule, e.g., dsRNA) comprises a 5′ phosphate group. For example, the nucleic acid molecule can be phosphorylated, e.g., by T4 polynucleotide kinase, prior to immobilization.

The immobilization (e.g., coupling) reaction can be performed in the presence of one or more cross-linkers. Exemplary crosslinkers include, but not limited to, 1-Ethyl-3-(3-dimethylaminopropyl)carbodiimide hydrochloride (EDC) and imidazole. A typical reaction buffer can contain, e.g., 100 mM Na₃PO₄, 1.5 M NaCl, 100 mM EDTA, at pH7.0. The reaction mixture can be incubated between about 25° C. and about 65° C., e.g., between about 35° C. and about 55° C., e.g., at about 37° C. or about 50° C.

After immobilization of the nucleic acid molecules (e.g., double-stranded oligonucleotide or RNA molecule, e.g., dsRNA), the plate can be washed one or more times using wash buffer, reaction buffer, and/or PBS. A typical wash buffer can contain, e.g., 5× SSC with 0.25% SDS (in distilled H₂O). After washing, the plate can be keep at 4° C. for future use.

### Anti-Nucleic Acid Antibody Assays

Various assays (e.g., immunoassays) can be used in accordance with the methods and compositions of the invention to detect antibodies to nucleic acid (e.g., an oligonucleotide molecule (e.g., a double-stranded oligonucleotide), or an RNA molecule, e.g., a double-stranded RNA (dsRNA)). In one embodiment, the assay is an enzyme-linked immunosorbent assay (ELISA).

Methods to perform ELISA are described in the art, e.g., in Engvall and Perlman (1971) Immunochemistry 8 (9): 871-8744 and Van Weemen and Schuurs (1971) FEBS Letters 15 (3): 232-236. Traditional ELISA typically involves chromogenic reporters and substrates that produce observable color change to indicate the presence of antigen or analyte. Other ELISA or ELISA-like techniques can use fluorogenic, electrochemiluminescent, and quantitative PCR reporters to create quantifiable signals, e.g., to achieve higher sensitivities and multiplexing. These ELISA techniques are described, e.g., in Leng et al. (2008) J Gerontol a Biol Sci Med Sci 63 (8): 879-884; Richter (2004) Chem Rev, 104, 3003-36.and Niemeyer et al. (2007) Nat Protoc 2: 1918-30. Although assays of this type use a nonenzymatic reporter, given that the general principles in these assays are largely similar, they are grouped in the same category as ELISAs.

The types of ELISA include, but not limited to, indirect ELISA, sandwich ELISA, competitive ELISA, and multiple and portable ELISA.

### Indirect ELISA

A typical indirect ELISA can be performed as follows.

A buffered solution of the antigen is first added and immobilized to each well of a microtiter plate. A solution of nonreacting protein, e.g., bovine serum albumin or casein, is added to the well. Next, a primary antibody is added, which binds specifically to the antigen coating the well. This primary antibody can also be in the serum of a donor to be tested for reactivity towards the antigen. Then, a secondary antibody is added, which will bind the primary antibody. In one embodiment, the secondary antibody can have an enzyme attached to it, which has a negligible effect on the binding properties of the antibody. In another embodiment, the primary antibody itself is conjugated to the enzyme. The enzyme can act as an amplifier, for example, producing more signal molecules even if only few enzyme-linked antibodies remain bound. A substrate for this enzyme is then added and the substrate changes color upon reaction with the enzyme. This color change shows the binding of the secondary antibody to the primary antibody and/or the binding of the primary antibody to the antigen, which indicates that the donor has an immune reaction to the antigen. The higher the concentration of the primary antibody is present in the serum, the stronger the color changes. In some embodiments, a spectrometer is used to give quantitative values for color strength.

### Sandwich ELISA

A typical sandwich ELISA can be performed as follows.

A microtiter plate surface is prepared to which a known quantity of capture antibody is bound. Any nonspecific binding sites on the surface are blocked and the antigen is applied to the plate. The plate is then washed to remove unbound antigen. Next, a specific antibody (or a sample containing the specific antibody) is added and binds to antigen (hence the “sandwich”: the antigen is stuck between two antibodies). Without the first layer of “capture” antibody, any proteins in the sample (including serum proteins) may competitively adsorb to the plate surface, lowering the quantity of antigen immobilized. Use of the purified specific antibody to attach the antigen to the plastic can eliminate a need to purify the antigen (e.g., from complicated mixtures before the measurement), simplifying the assay, and increasing the specificity and the sensitivity of the assay.

Next, an enzyme-linked secondary antibody is applied as a detection antibody that also binds specifically to the antibody, e.g., the Fc region. The plate is washed to remove the unbound antibody-enzyme conjugates. By using an enzyme-linked secondary antibody that binds the Fc region of other antibodies, this same enzyme-linked antibody can be used to detect antibodies from various sources. A chemical is then added to be converted by the enzyme into a color or fluorescent or electrochemical signal. The absorbency or fluorescence or electrochemical signal (e.g., current) of the plate wells is measured to determine the presence and quantity of antigen.

### Competitive ELISA

For the detection of ADAs, a typical competitive ELISA can be performed as follows.

A microtiter plate is coated with the antigen. Two specific antibodies are used, one conjugated with enzyme and the other present in the sample (if the sample is positive for the antibody). Cumulative competition occurs between the two antibodies for the same antigen, causing a stronger signal to be seen. The sample to be tested is added to the plate and incubated (e.g., at 37° C.) and then washed. If the antibodies are present, the antigen-antibody reaction occurs and no antigen is left for the enzyme-labeled antibodies. These enzyme-labeled antibodies remain free upon addition and are washed off during washing. Next, a substrate is added and remaining enzymes elicit a chromogenic or fluorescent signal. The positive result shows no or less color change because there is no or less enzyme to act on it.

### Multiple and Portable ELISA

Multiple and portable ELISA uses a solid phase made up of an immunosorbent polystyrene rod with eight to twelve protruding ogives. The entire device is immersed in a test tube containing the collected sample and the following steps (washing, incubation in conjugate and incubation in chromogens) are carried out by dipping the ogives in microwells of standard microplates filled with reagents.

The advantages of this technique include, e.g., the ogives can each be sensitized to a different reagent, allowing the simultaneous detection of different antibodies and/or different antigens for multiple-target assays; the sample volume can be increased to improve the test sensitivity in clinical samples; one ogive is left unsensitized to measure the nonspecific reactions of the sample; and the use of laboratory supplies for dispensing sample aliquots, washing solution and reagents in microwells is not required, facilitating the development of ready-to-use lab kits and on-site testing.

Multiple and portable ELISA are described, e.g., in U.S. Pat. No. 7,510,687 and EP 1499894 B1

The assays described herein are scored (as positive or negative or quantity) according to standard methods known to those of skill in the art. The particular method of scoring will depend on the assay format and choice of label. For example, ELISA can be run in a qualitative or quantitative format. Qualitative results provide a simple positive or negative result (yes or no) for a sample. The cutoff between positive and negative can be determined by empirical or statistical analysis. For example, two or three times the standard deviation (error inherent in a test) can be used to distinguish positive from negative samples. In quantitative ELISA, the optical density (OD) of the sample is compared to a standard curve, which is typically a serial dilution of a known-concentration solution of the target molecule. For example, if a test sample returns an OD of 1.0, the point on the standard curve that gave OD=1.0 must be of the same analyte concentration as the sample.

The term “labeled” is intended to encompass direct labeling of an antibody by coupling (i.e., physically linking) a detectable substance to the antibody, as well as indirect labeling of the antibody by reactivity with another reagent that is directly labeled. Examples of indirect labeling include detection of a primary antibody using a fluorescently labeled secondary antibody.

In one embodiment, the secondary antibody is labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair (e.g., biotin-streptavidin)), or an antibody fragment (e.g., a single-chain antibody, an isolated antibody hypervariable domain, etc.), is used.

One can immobilize either the probe, e.g., antigen (e.g., the nucleic acid molecule) or the antibody, on a solid support. For example, the antigen or the antibody can be anchored onto a solid phase support, also referred to as a substrate, and detecting target complexes anchored on the solid phase at the end of the reaction. In one embodiment of such a method, a sample from a subject, which is to be assayed for presence and/or concentration of an antibody, can be anchored onto a carrier or solid phase support. In another embodiment, the reverse situation is possible, in which the probe can be anchored to a solid phase and a sample from a subject can be allowed to react as an unanchored component of the assay.

Many methods for anchoring assay components to a solid phase are known in the art. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

In order to conduct assays with the above-mentioned approaches, the non-immobilized component is added to the solid phase upon which the second component is anchored. After the reaction is complete, uncomplexed components can be removed (e.g., by washing) under conditions such that any complexes formed will remain immobilized upon the solid phase. The detection of marker/probe complexes anchored to the solid phase can be accomplished in a number of methods outlined herein.

Other suitable carriers for binding antibody or antigen are known in the art. For example, antigens can be immobilized onto a solid phase support. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

The assays described herein can use a “capture agent” to specifically bind to and often immobilize the antigen or analyte. The capture agent is a moiety that specifically binds to the antigen or analyte. In another embodiment, the capture agent is an antibody that specifically binds a nucleic acid (e.g., RNA, e.g., siRNA) molecule. The antibody can be produced by any of a number of means known to those of skill in the art.

Immunoassays also often utilize a labeling agent to specifically bind to and label the binding complex formed by the capture agent or antigen (e.g., the RNA molecule) and the analyte (e.g., the anti-drug antibody). The labeling agent can itself be one of the moieties comprising the antibody/analyte complex. Thus, the labeling agent can be a labeled polypeptide or a labeled anti-antibody. Alternatively, the labeling agent can be a third moiety, such as another antibody, that specifically binds to the antibody/polypeptide complex.

In one embodiment, the labeling agent is a second antibody bearing a label. Alternatively, the second antibody can lack a label, but it can, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin. Detection can be facilitated by coupling the antibody to a detectable substance. Examples of detectable substances include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials include, but are not limited to, ¹²⁵I, ¹³¹I, ³⁵S or ³H.

Other proteins capable of specifically binding immunoglobulin constant regions, such as protein A or protein G can also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

As indicated above, immunoassays for the detection and/or quantification of anti-drug antibodies can take a wide variety of formats well known to those of skill in the art. Exemplary immunoassays for detecting an anti-drug antibody can be competitive or noncompetitive.

Antibodies for use in the various immunoassays described herein can be produced as described herein and the appended Examples.

### Additional Assay Formats

Additional methods and assay described herein include evaluation of an antibody against a nucleic acid molecule (e.g., an oligonucleotide molecule (e.g., a double-stranded oligonucleotide), or an RNA molecule, e.g., a double-stranded RNA (dsRNA)), e.g., an anti-drug antibody (ADA) in solution.

In one embodiment, the method or assay includes:

(a) providing the nucleic acid molecule (e.g., double-stranded oligonucleotide or RNA molecule, e.g., dsRNA);

(b) providing a pre-determined (e.g., known) amount of a binding agent, e.g., an antibody molecule, that binds to nucleic acid molecule (e.g., an antibody molecule as described herein), wherein either the nucleic acid molecule or the binding agent, or both are detectably labeled (e.g., radioactively- or fluorescently-labeled),

(c) combining, e.g., in solution, the nucleic acid molecule and the binding agent in the presence or the absence of a sample (e.g., a sample acquired from a subject) under conditions that allow binding of either the binding agent or the antibody, if present in the sample, to the nucleic acid molecule to occur.

In certain embodiments, the method further comprises determining the amount of a complex between the nucleic acid molecule and the binding agent, wherein a decrease in said complex is indicative of the level (e.g., presence or amount) of the antibody against the nucleic acid molecule in the sample. In certain embodiments, the amount of the complex between the nucleic acid molecule and the binding agent is determined as an inverse of the amount of the free nucleic acid molecule or the binding agent detected. For example, if the binding agent is detectably-labeled, the amount of free binding agent is indicative of the amount of the antibody to the nucleic acid molecule present in the sample.

In the aforesaid embodiments, the combining step is effected in solution, e.g., using a radioimmunoas say (RIA). Other alternative methods and assays for determining a binding interaction can be used, for example, Surface Plasmon Resonance (e.g., BIAcore).

In certain embodiments, the binding agent is an antibody molecule that that binds in a sequence-specific manner to an RNA molecule, e.g., a dsRNA. In other embodiments, the binding agent binds to a modified RNA molecule, e.g., a fluoro group (e.g., a fluoro group in the 2′-position of a ribonucleotide) of the RNA molecule; or a ligand in a conjugate of the RNA molecule, e.g., a ligand that includes one or more N-acetylgalactosamine (GalNAc) ligands. In certain embodiments, the binding agent is detectably-labeled (e.g., radioactively- or fluorescently-labeled).

In other embodiments, the probe (e.g., the nucleic acid molecule or capture antibody), when it is the unanchored assay component or in solution, can be labeled for the purpose of detection and readout of the assay, either directly or indirectly, with detectable labels discussed herein and which are known to one skilled in the art.

It is also possible to directly detect target antibody/probe complex formation without further manipulation or labeling of either component (target antibody or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a target antibody can be accomplished without labeling either assay component (probe or target antibody) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with target antibody and probe as solutes in a liquid phase. In such an assay, the complexed target antibody and probe are separated from uncomplexed components by any of a number of standard techniques, including but not limited to: differential centrifugation, chromatography, electrophoresis and immunoprecipitation. In differential centrifugation, marker/probe complexes can be separated from uncomplexed assay components through a series of centrifugal steps, due to the different sedimentation equilibria of complexes based on their different sizes and densities (see, for example, Rivas, G., and Minton, A.P., 1993, Trends Biochem Sci. 18(8):284-7). Standard chromatographic techniques can also be utilized to separate complexed molecules from uncomplexed ones. For example, gel filtration chromatography separates molecules based on size, and through the utilization of an appropriate gel filtration resin in a column format, for example, the relatively larger complex can be separated from the relatively smaller uncomplexed components. Similarly, the relatively different charge properties of the marker/probe complex as compared to the uncomplexed components can be exploited to differentiate the complex from uncomplexed components, for example, through the utilization of ion-exchange chromatography resins. Such resins and chromatographic techniques are well known to one skilled in the art (see, e.g., Heegaard, N.H., 1998, J. Mol. Recognit. Winter 11(1-6):141-8; Hage, D.S., and Tweed, S.A. J Chromatogr B Biomed Sci Appl 1997 Oct. 10; 699(1-2):499-525). Gel electrophoresis can also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typical. Appropriate conditions to the particular assay and components thereof will be well known to one skilled in the art.

### Kits

The invention also encompasses kits for detecting the presence of an anti-nucleic acid antibody, e.g., an anti-drug antibody, in a biological sample, e.g., a sample containing whole blood or serum. Such kits can be used to determine if a subject is suffering from the consequence or is at increased risk of developing anti-drug antibodies. For example, the kit can comprise a compound or agent capable of detecting an anti-drug antibody in a biological sample and means for determining the amount of the anti-drug antibody in the sample (e.g., a nucleic acid molecule described herein and a secondary antibody described herein). Kits can also include instructions for interpreting the results obtained using the kit.

### Antibody Molecules

A nucleic acid (e.g., an oligonucleotide molecule (e.g., a double-stranded oligonucleotide), or an RNA molecule, e.g., a double-stranded RNA (dsRNA)) molecule can be used as an immunogen to generate antibodies using standard techniques for polyclonal and monoclonal antibody preparation. These antibodies can be used, e.g., as positive controls and/or as capture agents in the assays to detect anti-drug antibodies. In certain embodiments, the antibody molecule binds in a sequence-specific manner to an RNA molecule, e.g., a dsRNA. In other embodiments, the antibody molecule binds to a modified RNA molecule, e.g., a fluoro group (e.g., a fluoro group in the 2′-position of a ribonucleotide) of the RNA molecule; or a ligand in a conjugate of the RNA molecule, e.g., a ligand that includes one or more N-acetylgalactosamine (GalNAc) ligands. In certain embodiments, the antibody molecule binds is detectably-labeled (e.g., radioactively- or fluorescently-labeled as described herein).

An immunogen typically is used to prepare antibodies by immunizing a suitable (i.e., immunocompetent) subject such as a rabbit, goat, mouse, or other mammal or vertebrate. An appropriate immunogenic preparation can contain, for example, recombinantly-expressed or chemically-synthesized nucleic acids. The preparation can further include an adjuvant, such as Freund's complete or incomplete adjuvant, or a similar immunostimulatory agent.

Accordingly, another aspect of the invention pertains to antibodies directed against a polypeptide of the invention. The terms “antibody” and “antibody substance” as used interchangeably herein refer to immunoglobulin molecules and immunologically active portions of immunoglobulin molecules, i.e., molecules that contain an antigen binding site which specifically binds an antigen, such as a nucleic acid molecule (e.g., double-stranded oligonucleotide or RNA molecule, e.g., dsRNA). A molecule which specifically binds to a given nucleic acid molecule (e.g., double-stranded oligonucleotide or RNA molecule, e.g., dsRNA) is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a double stranded oligonucleotide or nucleic acid molecule (e.g., double-stranded oligonucleotide or RNA molecule, e.g., dsRNA) as an immunogen. Antibody-producing cells can be obtained from the subject and used to prepare monoclonal antibodies by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the antigen of interest, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with the antigen of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions can be made using standard recombinant DNA techniques. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies can be produced using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Freemont, Calif.), can be engaged to provide human antibodies directed against a selected antigen using technology similar to that described above.

Detection can be facilitated by coupling the antibody to a detectable substance. Examples of detectable substances include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials include, but are not limited to, ¹²⁵I, ¹³¹I, ³⁵S or ³H.

### EXAMPLES

The following examples illustrate the methods of RNA molecule (iRNA) synthesis, coating iRNA onto plates, generation and characterization of control antibodies, and assays for measuring antibodies against RNA molecules (iRNA). The assays, methods, and compositions described herein allow for detecting various anti-drug antibodies against different epitopes of nucleic acid (e.g., RNA, e.g., iRNA) molecules.

### Example 1. siRNA Synthesis

### Source of Reagents

Where the source of a reagent is not specifically given herein, such reagent may be obtained from any supplier of reagents for molecular biology at a quality/purity standard for application in molecular biology.

### Oligonucleotide Synthesis

All oligonucleotides are synthesized on an AKTAoligopilot synthesizer. Commercially available controlled pore glass solid support (dT-CPG, 500 Å, Prime Synthesis) and RNA phosphoramidites with standard protecting groups, 5′-O-dimethoxytrityl N6-benzoyl-2′-t-butyldimethylsilyl-adenosine-3′-O-N,N′-diisopropyl-2-cyanoethylphosphoramidite, 5′-O-dimethoxytrityl-N4-acetyl-2′-t-butyldimethylsilyl-cytidine-3′-O-N,N′-diisopropyl-2-cyanoethylphosphoramidite, 5′-O-dimethoxytrityl-N2-isobutryl-2′-t-butyldimethylsilyl-guanosine-3′-O-N,N′-diisopropyl-2-cyanoethylphosphoramidite, and 5′-O-dimethoxytrityl-2′-t-butyldimethylsilyl-uridine-3′-O-N,N′-diisopropyl-2-cyanoethylphosphoramidite (Pierce Nucleic Acids Technologies) were used for the oligonucleotide synthesis. The 2′-F phosphoramidites, 5′-O-dimethoxytrityl-N4-acetyl-2′-fluro-cytidine-3′-O-N,N′-diisopropyl-2-cyanoethyl-phosphoramidite and 5′-O-dimethoxytrityl-2′-fluro-uridine-3′-O-N,N′-diisopropyl-2-cyanoethyl-phosphoramidite are purchased from (Promega). All phosphoramidites are used at a concentration of 0.2M in acetonitrile (CH₃CN) except for guanosine which is used at 0.2M concentration in 10% THF/ANC (v/v). Coupling/recycling time of 16 minutes is used. The activator is 5-ethyl thiotetrazole (0.75M, American International Chemicals); for the PO-oxidation iodine/water/pyridine is used and for the PS-oxidation PADS (2%) in 2,6-lutidine/ACN (1:1 v/v) is used.

3′-ligand conjugated strands are synthesized using solid support containing the corresponding ligand. For example, the introduction of cholesterol unit in the sequence is performed from a hydroxyprolinol-cholesterol phosphoramidite. Cholesterol is tethered to trans-4-hydroxyprolinol via a 6-aminohexanoate linkage to obtain a hydroxyprolinol-cholesterol moiety. 5′-end Cy-3 and Cy-5.5 (fluorophore) labeled iRNAs are synthesized from the corresponding Quasar-570 (Cy-3) phosphoramidite are purchased from Biosearch Technologies. Conjugation of ligands to 5′-end and or internal position is achieved by using appropriately protected ligand-phosphoramidite building block. An extended 15 min coupling of 0.1 M solution of phosphoramidite in anhydrous CH₃CN in the presence of 5-(ethylthio)-1H-tetrazole activator to a solid-support-bound oligonucleotide. Oxidation of the internucleotide phosphite to the phosphate is carried out using standard iodine-water as reported (1) or by treatment with tert-butyl hydroperoxide/acetonitrile/water (10:87:3) with 10 min oxidation wait time conjugated oligonucleotide. Phosphorothioate is introduced by the oxidation of phosphite to phosphorothioate by using a sulfur transfer reagent such as DDTT (purchased from AM Chemicals), PADS and or Beaucage reagent. The cholesterol phosphoramidite is synthesized in house and used at a concentration of 0.1 M in dichloromethane. Coupling time for the cholesterol phosphoramidite is 16 minutes.

### Deprotection I (Nucleobase Deprotection)

After completion of synthesis, the support is transferred to a 100 mL glass bottle (VWR). The oligonucleotide is cleaved from the support with simultaneous deprotection of base and phosphate groups with 80 mL of a mixture of ethanolic ammonia [ammonia: ethanol (3:1)] for 6.5 h at 55° C. The bottle is cooled briefly on ice and then the ethanolic ammonia mixture is filtered into a new 250-mL bottle. The CPG is washed with 2×40 mL portions of ethanol/water (1:1 v/v). The volume of the mixture is then reduced to ˜30 mL by roto-vap. The mixture is then frozen on dry ice and dried under vacuum on a speed vac.

### Deprotection II (Removal of 2′-TBDMS Group)

The dried residue is resuspended in 26 mL of triethylamine, triethylamine trihydrofluoride (TEA●3HF) or pyridine-HF and DMSO (3:4:6) and heated at 60° C. for 90 minutes to remove the tert-butyldimethylsilyl (TBDMS) groups at the 2′ position. The reaction is then quenched with 50 mL of 20 mM sodium acetate and the pH is adjusted to 6.5. Oligonucleotide is stored in a freezer until purification.

### Analysis

The oligonucleotides are analyzed by high-performance liquid chromatography (HPLC) prior to purification and selection of buffer and column depends on nature of the sequence and or conjugated ligand.

### HPLC Purification

The ligand-conjugated oligonucleotides are purified by reverse-phase preparative HPLC. The unconjugated oligonucleotides are purified by anion-exchange HPLC on a TSK gel column packed in house. The buffers are 20 mM sodium phosphate (pH 8.5) in 10% CH₃CN (buffer A) and 20 mM sodium phosphate (pH 8.5) in 10% CH₃CN, 1M NaBr (buffer B). Fractions containing full-length oligonucleotides are pooled, desalted, and lyophilized. Approximately 0.15 OD of desalted oligonucleotides are diluted in water to 150 μL and then pipetted into special vials for CGE and LC/MS analysis. Compounds are then analyzed by LC-ESMS and CGE.

iRNA Preparation

For the general preparation of siRNA, equimolar amounts of sense and antisense strand are heated in 1xPBS at 95° C. for 5 min and slowly cooled to room temperature. Integrity of the duplex is confirmed by HPLC analysis.

Nucleic acid sequences are represented below using standard nomenclature, and specifically the abbreviations of Table 1. It will be understood that the monomers shown in Table 1, when present in an oligonucleotide, are mutually linked by 5′-3′-phosphodiester bonds.

TABLE 1
Abbreviations of nucleotide monomers used in nucleic acid sequence representation

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| (Chd) | 2′-O-hexadecyl-cytidine-3′-phosphate |
| (Chds) | 2′-O-hexadecyl-cytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tb | beta-L-thymidine-3′-phosphate |
| Tbs | beta-L-thymidine-3′-phosphorothioate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Ub | beta-L-uridine-3′-phosphate |
| Ubs | beta-L-uridine-3′-phosphorothioate |
| Uf | 2′-fluorouridine-3′-phosphate |
| Ufs | 2′-fluorouridine -3′-phosphorothioate |
| (Uhd) | 2′-O-hexadecyl-uridine-3′-phosphate |
| (Uhds) | 2′-O-hexadecyl-uridine-3′-phosphorothioate |
| Us | uridine-3′-phosphorothioate |
| N | any nucleotide (G, A, C, T or U) |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine |
| dTs | 2′-deoxythymidine-3′-phosphorothioate |
| dU | 2′-deoxyuridine |
| s | phosphorothioate linkage |
| L96¹ | N-[tris(GalNAc-alkyl)-amidodecanoyl)]-4-hydroxyprolinol Hyp-(GalNAc-alkyl)3 |
| (Aeo) | 2′-O-methoxyethyladenosine-3′-phosphate |
| (Aeos) | 2′-O-methoxyethyladenosine-3′-phosphorothioate |
| (Geo) | 2′-O-methoxyethylguanosine-3′-phosphate |
| (Geos) | 2′-O-methoxyethylguanosine-3′-phosphorothioate |
| (Teo) | 2′-O-methoxyethyl-5-methyluridine-3′-phosphate |
| (Teos) | 2′-O-methoxyethyl-5-methyluridine-3′-phosphorothioate |
| (m5Ceo) | 2′-O-methoxyethyl-5-methylcytidine-3′-phosphate |
| (m5Ceos) | 2′-O-methoxyethyl-5-methylcytidine-3′-phosphorothioate |

¹The chemical structure of L96 is as follows:

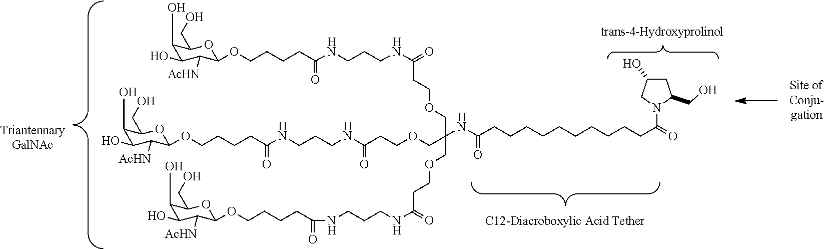

### Example 2. Strategies for Developing Multi-Tiered ADA Assays for RNA Molecules

The development of multi-tiered anti-drug antibody (ADA) assays for iRNAs allows for evaluation of antibody response after drug administration. The multi-tiered ADA assay described herein can include, e.g., a screening assay, a confirmation assay, and a titration assay.

Screening Assay

The screening assay can be used to identify potentially positive samples. Assay cut-point (CP) can be determined during validation to detect 5% false positives (Mire-Sluis AR et al. J Immunol Methods. 2004; 289(1-2):1-16). Cut-point is the level of response (OD at A450) at or above which a sample is defined as positive and below which is defined as negative. To establish cut-point, about 15 non-clinical samples or at least 50 clinical samples are needed.

### Confirmation Assay

The confirmation assay can be used to identify true positive samples by spiking with drug prior to assay. Drug competition (e.g., immunodepletion/competitive inhibition) can be used to determine the percent inhibition by drug. Percent inhibition necessary to identify true positive is called confirmatory cut-point (CCP). CCP is determined during validation and false positive samples will be identified.

### Titration Assay

If necessary, the titration assay can be used to determine the titer of each positive sample. Titration can be done by dilution of serum that gives positive signal.

As described below, to develop a multi-tiered ADA assay for two siRNA drugs, AD-59153 and ALN-AD-59155, control antibody reagents against those two drug compounds were generated. In addition, the siRNA drug compounds were properly coated on plates to facilitate antibody binding and subsequent detection.

### Example 3. Coupling of Phosphorylated iRNAs to Plates

iRNA compounds were covalently coupled to CovaLink™ NH modules/strip plates (Nalge-Nunc) through the 5′ phosphate groups of the duplexes.

Phosphorvlation of siRNA Conjugates

To add 5′-phosphate to the sense strand and/or antisense strand of the iRNA conjugate, the siRNA duplex, sense strand, and antisense strand were individually phosphorylated by T4 polynucleotide kinase. After phosphorylation, the siRNA duplex had both the sense and antisense strands phosphorylated. The 5′ phosphorylated sense strand was denatured and then annealed with the non-phosphorylated complementary strand to produce the siRNA duplex that only has the sense strand phosphorylated. Similarly, the 5′ phosphorylated antisense strand was denatured and then annealed with the non-phosphorylated complementary strand to produce the siRNA duplex that only has the antisense strand phosphorylated. The phosphorylated siRNA duplex, sense strand, and antisense strand were purified and desalted (e.g., to remove Tris).

FIG. 2A is a schematic representation of the 5′ phosphorylation of the siRNA duplex, sense strand, and antisense strand. The sense strand of the iRNA duplex contains a GalNAc moiety at the 3′ end.

The amount of phosphorylated iRNA duplexes covalently coupled to the plate was quantified by RT-qPCR. The coating conditions (e.g., reaction buffer, input, incubation temperature, incubation time, and washing) were optimized.

Covalently Coupling Phosphorylated siRNA Conjugates to Plates

FIG. 2B shows how the phosphorylated iRNA conjugates can be covalently coupled to the plates through the 5′ phosphate groups of the duplex. As shown in FIG. 2B, the phosphorylated iRNA conjugates can be covalently coupled to the plates at the 5′ end of the sense strand, 5′ end of the antisense strand, or both.

The phosphorylated iRNA conjugates were coupled to the plates (CovaLink™) through the linkers shown in FIG. 3. The linker contains a secondary amino group positioned at the end of the spacer arm. The linkers were grafted onto the plates at a density of approximately 10¹²/mm².

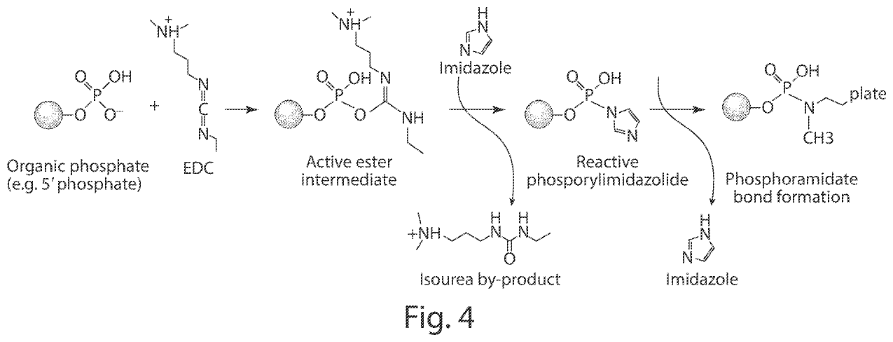

FIG. 4 is a schematic representation of the coupling reaction. 5′-phosphorylated GalNAc-iRNA conjugates were coupled to the secondary amino groups positioned at the end of spacer arms covalently grafted to the polystyrene surface, using cross-linker 1-Ethyl-3-(3-dimethylaminopropyl)carbodiimide hydrochloride (EDC) and imidazole.

Coating conditions, including, but not limited to, reaction buffer, input, incubation temperature, incubation time and washing, were optimized. An exemplary protocol for plate coating is provided below.

### Protocol for Plate Coating

### Materials

Covalink™ NH modules/strip plates (Product No. 478042) and 1-Ethyl-3-(3-dimethylaminopropyl)carbodiimide hydrochloride (EDC), 25 g (Product No. 22981) were purchased from Thermo Scientific. 1-methylimidazole (Product No. 478042 336092-1L), SSC buffer 20× concentrate (Product No. 478042 S6639-1L), EDTA (Product No. 478042 E6758-500G), and Triton X-100 (Product No. 478042 T8787-250 mL) were from Sigma. NaCl (Product No. 478042 SX0420-3) was from EMD. Na₃PO₄.12H₂O (Product No. 478042 3836-05) was from J.T. Baker. 10× PBS buffer, pH 7.4 (Catalog No. AM9625) was from Ambion.

The double stranded oligonucleotides or nucleotide sequences for the sense and antisense strands of the siRNA duplexes used in the Examples are shown in Table 2.

TABLE 2
Nucleotide sequences for (AD-59153) and
(AD-59155)

| | Sense | Antisense |
|---|---|---|
| Duplex | Strand Sequence | Strand Sequence |
| AD-59153 | 5′-PUfgGfgAfuUfu | 5′-uCfuUfgGfUfUfa |
| | CfAfUfgUfaacCfaA | CfaugAfaAfuCfcCfa |
| | fgAfL96-3′ | sUfsc-3′ |
| | (SEQ ID NO: 6) | (SEQ ID NO: 7) |
| (AD-59155) | 5'-PGfsgsUfuAfaC | 5′-usUfsgAfaGfuAf |
| | faCfCfAfuUfuAfcU | aAfuggUfgUfuAfaCf |
| | fuCfaAfL96-3′ | csasg-3′ |
| | (SEQ ID NO: 8) | (SEQ ID NO: 9) |
| AD-57740 | 5′-cuuAcGcuGAGuA | 5′-PUCGAAGuACUcAG |
| (Luc) | cuucGAdTsdT-3′ | CGuAAGdTsdT-3′ |
| | (SEQ ID NO: 10) | (SEQ ID NO: 11) |

Coding for the modifications in the above sequences: lowercase: 2′-OMe; d: 2′-deoxy; s: phosphorothioate; f: 2′ -fluoro; L96: N-[tris(GalNAc-alkyl)-amidodecanoyl)]-4-hydroxyprolinol Hyp-(GalNAc-alkyl)₃

### Reagents

10× reaction buffer was prepared by mixing 100 mM Na₃PO₄, 1.5 M NaCl, 100 mM EDTA, pH 7.0. 100 mM 1-methylimidazole (1-MeIm₇) was prepared using 1× reaction buffer. 0.1 μg/μL of phosphorylated duplex (AD-59153 or AD-59155) was reconstituted in nuclease-free H₂O. 0.2 M EDC was freshly made in 10 mM 1-MeIm₇, which was diluted from 100 mM 1-MeIm₇with 1× reaction buffer. Wash buffer was prepared by mixing 5× SSC with 0.25% SDS (in distilled H₂O). Coupling mix (scale up based on reaction numbers, one well per reaction for coupling) was prepared according to the recipe shown in Table 3.

TABLE 3
Coupling reaction mixture

| Coupling mix (for 1x reaction) | Volume (μL) |
|---|---|
| AD-59155 or AD-59153 (0.1 μg/μL) | 6 |
| 100 mM 1-MeIm₇ | 7.5 |
| 10x reaction buffer | 7.5 |
| Nuclease-free H₂O | 54 |
| Total | 75 |

### Procedures

To coat plates with phosphorylated duplex, 75 μL of coupling mix and 25 μL of freshly prepared 0.2 M EDC were aliquoted to each well (for negative controls, add 25 μL of 10 mM 1-MeIm₇ instead of 0.2 M EDC). Plates were sealed and incubated 37° C. (for AD-59155) or 50° C. (for AD-59153) for 24 hours.

Wash buffer was pre-warmed at 37° C. (for AD-59155) or 50° C. (for AD-59153). Plates were washed with 200 μL pre-warmed wash buffer (each well) with 5× repeats for a total of six washes, 2× washes with 200 μL 1× reaction buffer, followed by two more washes with 1× PBS. Plates were sealed and kept at 4° C. for future use.

To quantify the amount of coupled phosphorylated siRNA by reverse transcription based-quantitative PCR (RT-qPCR), 120 μL of 0.25% Triton X-100 (in PBS) was aliquoted to each well that was subjected to quantification. The plates were sealed and incubated at 95° C. for 6 min. 60 μL of solution from each well was obtained for RT-qPCR, while the plates/strips were in the 95° C. heat block.

Quantification of Coupled iRNA Conjugates

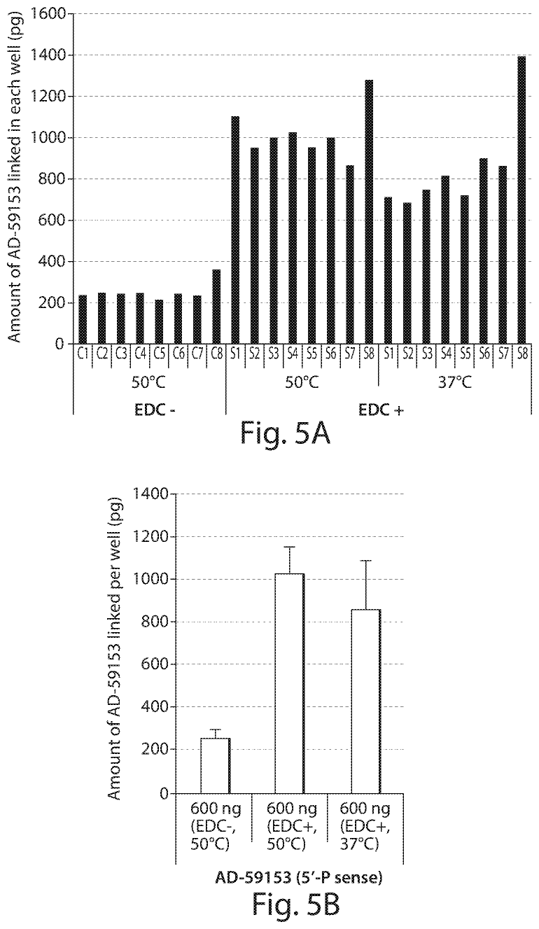

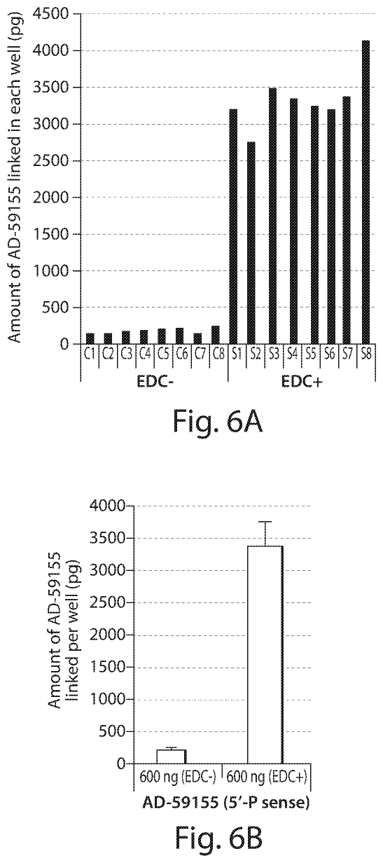

The amount of iRNA conjugates coupled to the plates was quantified by RT-qPCR. The results are shown in FIGS. 5A-6B.

As shown in FIGS. 5A-5B, AD-59153 was successfully coupled to the CovaLink™ plate after incubation at 50° C. and 37° C. in the presence of EDC. On average, approximately 1 ng (about 3.70×10¹⁰ molecules) of AD-59153 was coupled to each well. 600 ng of AD-59153 having a 5′ phosphorylated sense strand (and a non-5′-phosphorylated antisense strand) was used in the coupling reaction.

As shown in FIGS. 6A-6B, AD-59155 was also successfully coupled to the CovaLink™ plate in the presence of EDC. On average, approximately 3.4 ng (about 1.25×10¹¹ molecules) of AD-59155 was coupled to each well. 600 ng of AD-59155 having a 5′ phosphorylated sense strand (and a non-5′-phosphorylated antisense strand) was used in the coupling reaction.

The phosphorylated AD-59153 and AD-59155 were covalently cross-linked to the CovaLink™ plates in an input-dependent manner (data not shown).

### Example 4. Generation of Polyclonal Antibodies to iRNA Drug Compounds

This example illustrates the production and characterization of positive control antibodies for direct binding ADA assays (e.g., ELISA).

### General Schedule of Antibody Generation and Screening

The following three compounds were used for antibody generation: AD-59155, KLH-AD-59155 and KLH-AD-59153. The sense and antisense strand nucleotide sequences for AD-59155 and AD-59153 are provided in Example 3. Keyhole limpet hemocyanin (KLH) is a carrier protein used to boost immune response. Freud's adjuvant was used to increase the possibility of generating antibodies.

Five rabbits and one llama were used for each compound. Rabbits were used to generate normal immunoglobulins. Llamas generate largely heavy chain only immunoglobulins, which are resistance to heat/dry and capable of refolding and maintaining activity. Llamas were used because they may be more sensitive to immunogen than rabbits and generate large amount of antibodies.

Rabbits were injected 0.5 mg and llamas were injected 2 mg each compound subcutaneously, at Days 1, 7, 14, 28, 56 and 84. For the first injection, the antigen (drug compound) was mixed (1:1) with complete Freund's adjuvant (CFA), and all the other injections were mixed with incomplete Freund's adjuvant. Complete Freund's adjuvant (Product # F5881) and incomplete Freund's adjuvant (Product # F5506) were purchased from Sigma. Each mL of complete Freund's adjuvant contains 1 mg of heat-killed and dried Mycobacterium tuberculosis (strain H37Ra, ATCC 25177), 0.85 mL paraffin oil and 0.15 mL of mannide monooleate. Each mL of incomplete Freund's adjuvant contains 0.85 mL of paraffin oil and 0.15 mL of mannide monooleate.

At Days 42, 70 and 98, animals were bled for screening for immune response. After Day 98, the animals were either maintained (e.g., kept alive with monthly boost and ELISA testing) or sacrificed.

### Screening Results for AD-59155

Rabbits did not show any immune response at Day 98, and the llama showed very weak response. On Day 100, the first boost injection was administered with 50% increase in dose and the injection route changed to 80% intramuscular (IM) and 20% intradermal (ID) administration. Antibody titers on Day 110 were significantly improved after the first boost injection. The second boost (Day 128) for rabbits was the same as the first boost. The second boost for llama was 4 mg in CFA and by 100% IM administration. The antibody titers are shown in Table 4.

TABLE 4
Antibody titers determined by ELISA
(using Ova-AD-59155 as screening antigen)

| | Rabbits | | | | | Llama |
|---|---|---|---|---|---|---|
| Time | #18269 | #18270 | #18271 | #18272 | #18273 | #708 |
| Pre-bleed | 1.00E+00 | 1.00E+00 | 1.00E+00 | 1.00E+00 | 1.00E+00 | 1.00E+00 |
| Day 70 | 1.00E+00 | 1.00E+00 | 1.00E+00 | 1.00E+00 | 1.00E+00 | 1.00E+00 |
| Day 98 | 1.00E+00 | 1.00E+00 | 1.00E+00 | 1.00E+00 | 1.00E+00 | 1.13E+02 |
| Day 110 | 9.63E+03 | 1.56E+03 | 4.17E+03 | 1.49E+04 | 1.46E+04 | 1.24E+04 |
| Day 139 | 7.84E+03 | 5.57E+04 | 1.10E+04 | 4.93E+04 | 4.96E+04 | 4.45E+02 |

Serum from rabbits #18270 and #18271 were screened in the plates coated with AD-59155. As shown in Table 5, the antibody titers were comparable to the titers observed in the screening using Ova- AD-59155 (1.00×10³). Rabbits #18270, #18272, and #18273 were sacrificed. Rabbits #18269 and #18271 were boosted monthly. The llama was released.

TABLE 5
Antibody titers determined by ELISA (using the plates
coated with AD-59155, samples were diluted 100x)

| | A 450 nm | |
|---|---|---|
| Time | Rabbit #18270 | Rabbit #18271 |
| Pre-bleed | 0.117 | 0.195 |
| Day 110 | 1.028 | 0.620 |

### Screening Results for KLH-AD-59155

On Day 42, rabbits displayed a strong immune response to KLH-AD-59155, whereas llama displayed a moderate response (at Day 52). The antibody titers are shown in Table 6.

TABLE 6
Antibody titers determined by ELISA
(using Ova-AD-59155 as screening antigen)

| | Rabbits | | | | | Llama |
|---|---|---|---|---|---|---|
| Time | #19176 | #19177 | #19178 | #19179 | #19190 | #715 |
| Pre-bleed | 1.00E+00 | 1.00E+00 | 1.00E+00 | 1.00E+00 | 1.00E+00 | 1.00E+00 |
| Day 42 | 3.53E+06 | 8.19E+05 | 1.64E+06 | 3.27E+06 | 2.60E+07 | 1.58E+03 |
| (rabbits) | | | | | | |
| Day 52 | | | | | | |
| (llama) | | | | | | |
| Day 70 | 9.90E+05 | 2.40E+05 | 1.68E+06 | 2.71E+05 | 1.35E+06 | 1.34E+03 |
| (rabbits) | | | | | | |
| Day 74 | | | | | | |
| (llama) | | | | | | |

As shown in Table 7, the plates covalently coupled with AD-59155 worked for screening of anti-KLH-AD-59155 sera. Rabbits #19176, #19178, and #19180 were sacrificed. Rabbits #19177 and #19179 were boosted monthly. The llama was released after terminal bleed.

TABLE 7
Antibody titers determined by ELISA (A450 nm, using the plates coated
with AD-59155, samples were diluted 1,000x)

| | Rabbits | | | | | Llama |
|---|---|---|---|---|---|---|
| Time | #19176 | #19177 | #19178 | #19179 | #19190 | #715 |
| Pre-bleed | 0.167 | 0.129 | 0.143 | 0.142 | 0.156 | 0.129 |
| Day 42 | 2.460 | 3.284 | 3.406 | 3.486 | 3.430 | 0.165 |
| (rabbits) | | | | | | |
| Day 52 | | | | | | |
| (llama) | | | | | | |

### Screening Results for KLH-AD-59153

On Day 30/42, rabbits and llama showed strong immune response to KLH-AD-59153. The antibody titers are shown in Table 8.

TABLE 8
Antibody titers determined by ELISA
(using Ova-AD-59153 as screening antigen)

| | Rabbits | | | | | Llama |
|---|---|---|---|---|---|---|
| Time | #19149 | #19150 | #19151 | #19152 | #19153 | #716 |
| Pre-bleed | 1.00E+00 | 1.00E+00 | 1.00E+00 | 1.00E+00 | 1.00E+00 | 1.00E+00 |
| Day 42 | 2.62E+05 | 5.10E+05 | 9.54E+05 | 6.48E+05 | 1.16E+06 | 3.15E+05 |
| (rabbits) | | | | | | |
| Day 30 | | | | | | |
| (llama) | | | | | | |
| Day 70 | 3.05E+05 | 2.32E+05 | 5.95E+05 | 1.93E+05 | 2.45E+05 | 4.14E+06 |
| (rabbits) | | | | | | |
| Day 52 | | | | | | |
| (llama) | | | | | | |

As shown in Table 9, the plates covalently coupled with AD-59153 worked for screening of anti-KLH-AD-59153 sera. Rabbits #19150, #19151, and #19152 were sacrificed. Rabbits #19149 and #19153 were boosted monthly. The llama was released after terminal bleed.

TABLE 9
Antibody titers determined by ELISA (A450 nm, using the plates coated
with AD-59153, samples were diluted 1,000x)

| | Rabbits | | | | | Llama |
|---|---|---|---|---|---|---|
| Time | #19149 | #19150 | #19151 | #19152 | #19153 | #716 |
| Pre-bleed | 0.175 | 0.165 | 0.230 | 0.195 | 0.129 | 0.132 |
| Day 42 | 0.760 | 2.818 | 3.333 | 2.103 | 2.794 | 3.463 |
| (rabbits) | | | | | | |
| Day 30 | | | | | | |
| (llama) | | | | | | |

### Example 5. ADA Assays for siRNA Drug Compounds

This example illustrates the development of ADA assays (ELISAs) for AD-59155 and AD-59153.

### ADA Assay for AD-59155

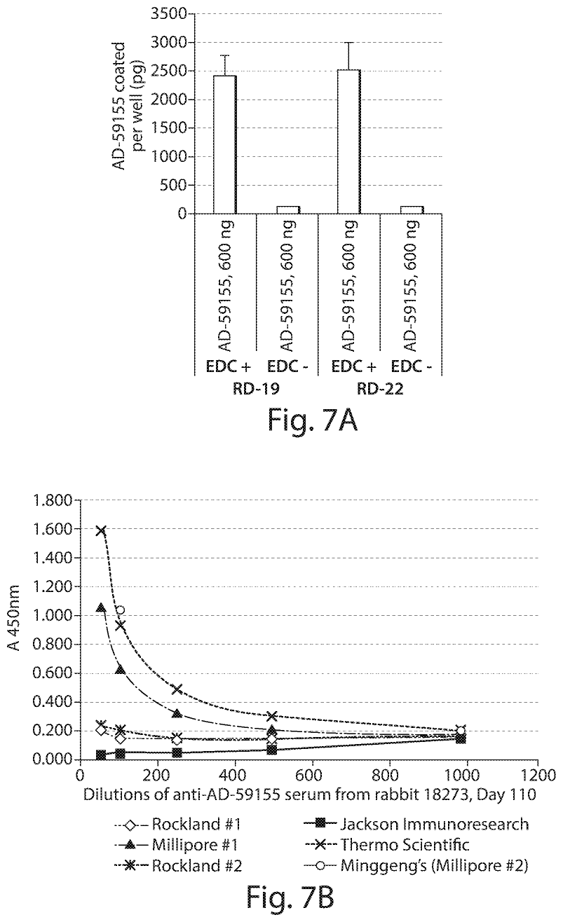

To develop an ADA assay for AD-59155, 600 ng of AD-59155 was used to coat each well of the plates as described in Example 3. FIG. 7A shows the amount of AD-59155 coated per well. As shown in FIG. 7A, AD-59155 was reliably coated onto the plates in the presence of EDC.

To identify a HRP conjugated secondary antibody for the ADA assay, various secondary antibodies were evaluated by ELISA using serial dilutions of anti-AD-59155 serum from rabbit #18273 on Day 110. Pooled normal monkey sera (1/100 in casein/TBS) were used for antiserum dilution. As shown in FIG. 7B, a HRP conjugated secondary antibody that can be used for detection of AD-59155 in the ADA assay was identified.

### ADA Assay for AD-59153

To examine the matrix effect on antibody detection, ELISA was performed using anti-KLH-AD-59153 serum from rabbit #19151, Day 42, serially diluted in either blocking buffer (casein/TBS) or pooled human sera (1/50 in blocking buffer).

A matrix effect is a consistent bias in analyte determinations between two sources of matrix, such as between serum and plasma, or serum and charcoal-stripped serum. It can be used to describe a known source of bias with an unknown cause. One important type of matrix effect is any that occurs between the matrix used to prepare the calibration curve, and the matrix of test samples.

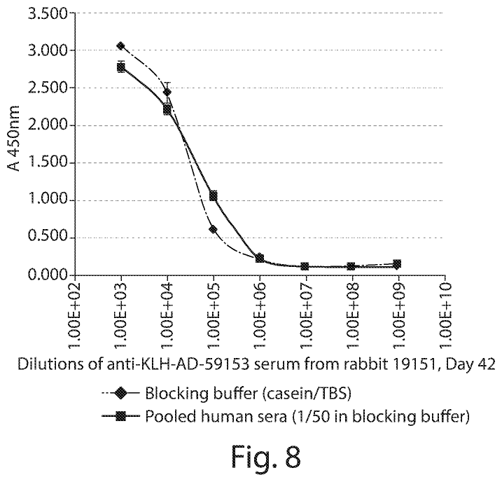

As shown in FIG. 8, the matrix effect on antibody detection is negligible and the assay sensitivity is not impacted.

Positive controls at different concentrations are useful for assay validation to determine precision, accuracy, selectivity, specificity, and reproducibility. To select the proper concentrations for positive controls, ELISA was performed using rabbit anti-KLH-AD-59153 serum (rabbit #19151, Day 70) serially diluted in pooled human serum (pre-diluted 100× in casein/TBS) in the plates coated with AD-59153. HRP-conjugated, goat anti-rabbit IgG (H+L) was diluted 250-fold.

The results are shown in Table 10.

TABLE 10
Evaluation of positive control at varying dilutions

| | A 450 nm | | |
|---|---|---|---|
| Dilutions (fold) | Average | Stdev | Selections |
| 1,000 | 3.290 | 0.001 | |
| 5,000 | 3.312 | 0.011 | |
| 10,000 | 3.317 | 0.020 | |
| 20,000 | 3.279 | 0.007 | |
| 40,000 | 3.281 | 0.014 | |
| 60,000 | 3.235 | 0.047 | |
| 80,000 | 3.131 | 0.074 | |
| 100,000 | 3.091 | 0.031 | HPC |
| 100,000 | 2.909 | 0.040 | |
| 200,000 | 1.761 | 0.019 | MPC |
| 500,000 | 0.830 | 0.010 | |
| 1,000,000 | 0.497 | 0.018 | LPC |
| 2,000,000 | 0.308 | 0.005 | LLPC |
| 5,000,000 | 0.201 | 0.006 | |
| 10,000,000 | 0.175 | 0.008 | |
| Matrix for dilution | 0.143 | 0.001 | |

HPC: high positive control;
MPC: medium positive control;
LPC: low positive control;
LLPC: lowest low positive control

Based on the results, positive controls at different dilution levels with the appropriate matrix were determined and used for the ADA assay development and validation.

### Protocol for ADA Assay

The ADA assay conditions, including wash buffer, blocking buffer, matrix MRD (minimum required dilution), control antibody dilutions, HRP-conjugated, secondary antibody, evaluated precision/reproducibility, were established. An exemplary protocol for the ADA assay (ELISA) is provided below.

### Materials

Plates were coated with AD-59155 or AD-59153. Casein in TBS (blocking buffer) was from Thermo scientific (Prod #37532). Goat Anti-rabbit IgG, HRP was from Millipore (Cat #12-348). Goat Anti-llama IgG—H&L, HRP was from Abcam (Cat# ab112786). TMB reagent was from Sigma (Cat # T0440). Other reagents include, e.g., Phosphate Buffered Saline (PBS), Tween-20, and 1M H₂SO₄. Wash buffer was prepared by mixing 0.1% Tween-20 in 1× PBS.

### Procedures

Plates were blocked with 160 μL blocking buffer each well at room temperature for 1 hour. Blocking buffer was subsequently removed (by tapping onto paper the paper towel three times)

Samples (e.g., serum samples) were prepared by dilution in blocking buffer. 100 μL sample was aliquoted in each well and incubated at 37° C. for 2 hours. The plates were washed five times with 160 μL wash buffer (by tapping onto the paper towel three times after each wash).

Secondary antibody (HRP-Conjugated, goat-anti-rabbit or llama IgG) was diluted 10,000 times with blocking buffer. 100 μL secondary antibody was added to each well and incubated at 37° C. for 1 hour. The plates were washed with 160 μL of Wash Buffer five times (tapping onto the paper towel 3× after each wash).

For detection, 100 μL TMB reagent was aliquoted to each well and incubated at 37° C. in dark for 10 minutes (until blue color developed). 100 μL 1 M H₂SO₄ was added to each to stop the reaction (color changed to yellow). Absorbance at 450 nm was measured using a plate reader.

### Example 5. Characterization of Control Antibodies

The specificity of antibodies against AD-59155 or AD-59153 was examined.

As described in more detail below, one of the anti-AD-59155 antibodies generated in the presence of Freund's adjuvant (from rabbit #18273) only recognized AD-59155 and four of the anti-KLH-AD-59155 antibodies only reacted with the sugar (GalNAc) part of the siRNA (AD-59155 or AD-59153) in an input-dependent fashion. Those antibodies can be used for ADA assay development for any other GalNAc-siRNA drugs. The anti-KLH-AD-59153 antibody from rabbit #19151 recognized both AD-59153 and AD-59155 compounds (2′-fluoro modified oligos) in an input-dependent fashion, indicating the antibody specifically recognizes the 2-fluoro carrying oligos. None of the anti-AD-59155, anti-KLH-AD-59155, and anti-KLH-AD-59153 antibodies reacted with phosphorylated Luc siRNA (AD-57740). These results suggest that antibody responses can be generated against any part of the drug product.

### Selectivity/Specificity of Polyclonal Anti-KLH-AD-59153 Antibodies

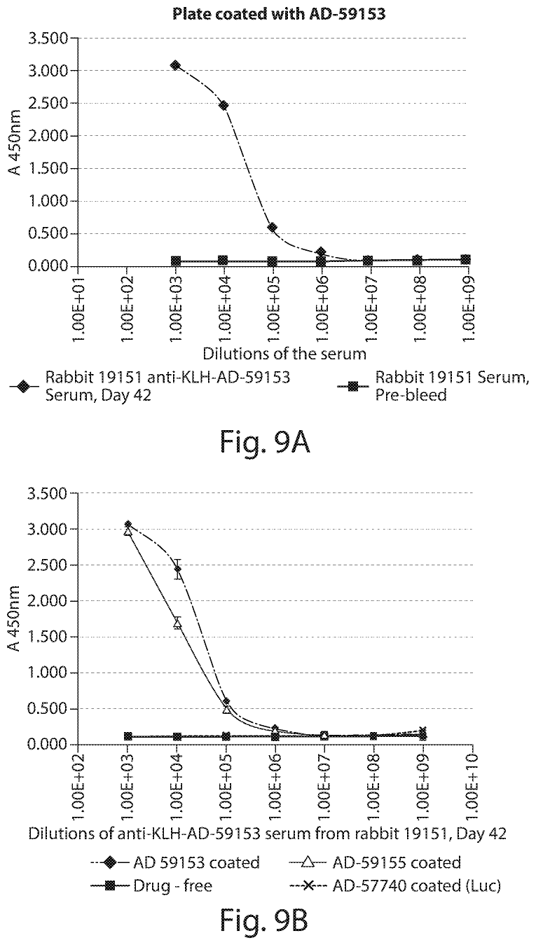

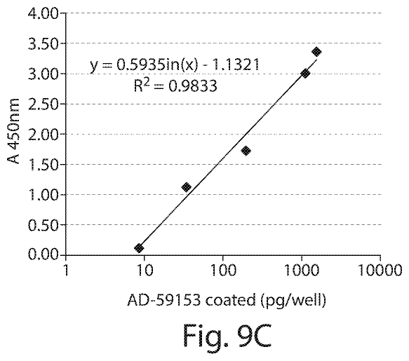

To test the selectivity/specific of polyclonal anti-KLH-AD-59153 antibodies, ELISA was performed in the plate coated with AD-59153, using serially diluted anti-KLH-AD-59153 serum from rabbit 19151, Day 42, or pre-bleed serum from the same rabbit. As shown in FIG. 9A, the anti-KLH-AD-59153 serum from rabbit 19151, Day 42 bound to the plate coated with AD-59153, but no binding was observed when the pre-bleed rabbit serum was used.

Next, ELISA was performed in the drug-free plate, or the plates coated with AD-59153, AD-59155, or AD-57740 (Luc). As shown in FIG. 9B, the anti-KLH-AD-59153 serum from rabbit #19151, Day 42 bound to the plates coated with AD-59153 and AD-59155, but not drug-free or AD-57740 (Luc) coated plates.

Further, ELISA was performed in the plates coated with varying amounts of AD-59153. Anti-KLH-AD-59153 antibody (rabbit #19151, Day 70) was diluted 10,000-fold. HRP-conjugated, goat anti-rabbit IgG (H+L) (Thermo Scientific) was diluted 5,000-fold. As shown in FIG. 9C, binding of the anti-AD-59153 antibodies to AD-59153-coated plate correlated with the amount of AD-59153 (with a coefficient of determination (R²)=0.9883).

### Selectivity/Specificity of Polyclonal Anti-KLH-AD-59155 Antibodies

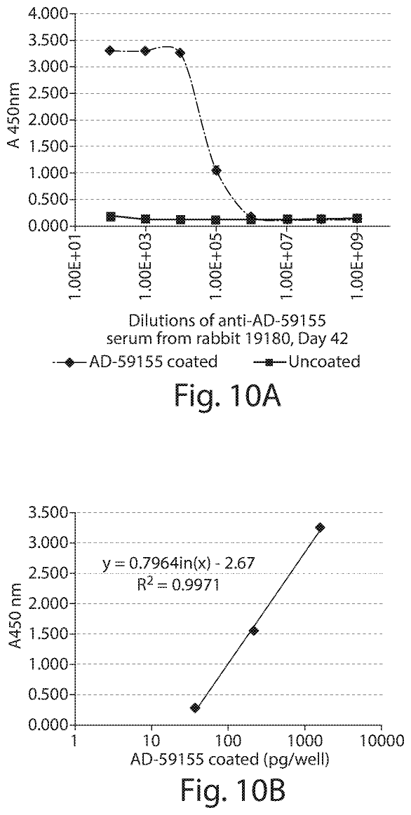

To test the selectivity/specific of polyclonal anti-KLH-AD-59155 antibodies, ELISA was performed in the uncoated plate or the plate coated with AD-59155. As shown in FIG. 10A, anti-KLH-AD-59155 serum from rabbit #19180, Day 42, bound to the plate coated with AD-59155 but not the uncoated plate.

Next, the ADA assay was performed in the plates coated with varying amounts of AD-59155. Anti-KLH-AD-59155 antibody (rabbit #19178, Day 70) was diluted 5,000-fold. HRP-conjugated, goat anti-rabbit IgG (H+L) (Thermo Scientific) was diluted 5,000-fold. As shown in FIG. 10B, binding of the anti-AD-59155 antibodies to AD-59155-coated plate correlated with the amount of AD-59155 (with a coefficient of determination (R²)=0.9971).

### Cross-Reactivity/Specificity of Anti-AD-59155 Antibodies

To examine the cross-reactivity/specificity of the anti-AD-59155 antibodies, ELISA was performed in the plates coated with different compounds.

Tested AD-59155 related compounds include: AD-59155, AD-59155 sugar-free (AD-61099), the phosphorylated sense strand of AD-59155 (A-119924), and the phosphorylated antisense strand of AD-59155 (A-119925). Tested AD-59153-related compounds include: AD-59153, AD-59153 sugar-free (AD-61007), the phosphorylated sense strand of AD-59153 (A-119922), and the phosphorylated antisense strand of AD-59153 (A-119923). Luc (AD-57740) was also tested.

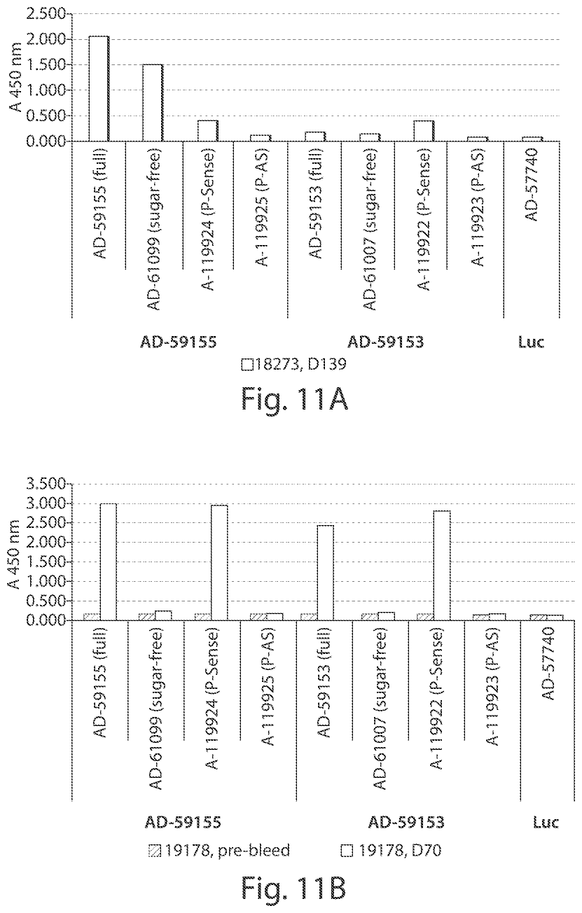

FIG. 11A shows the results when the polyclonal anti-AD-59155 antibodies from rabbit #18273 (Day 139) were used (diluted 500-fold). As shown in FIG. 11A, antibodies from this rabbit specifically recognized the duplexes of AD-59155 compounds (with or without sugar).

FIG. 11B shows the results when the polyclonal anti-AD-59155 antibodies from rabbit #19178 (Day 70) were used (diluted 10,000-fold). As shown in FIG. 11B, the antibodies from this rabbit (and three other rabbits in this group) only recognize the sugar moiety (GalNAc).

These results indicate that animals showed different antibody responses to the same antigen administrated by the same dosing regimen and route.

### Cross-Reactivity/Specificity of Anti-AD-59153 Antibodies

To examine the cross-reactivity/specificity of the anti-AD-59153 antibodies, the ADA assay was performed in the plates coated with different compounds. Tested AD-59155 related compounds include: AD-59155, AD-59155 sugar-free (AD-61099), the phosphorylated sense strand of AD-59155 (A-119924), and the phosphorylated antisense strand of AD-59155 (A-119925). Tested AD-59153 related compounds include: AD-59153, AD-59153 sugar-free (AD-61007), the phosphorylated sense strand of AD-59153 (A-119922), and the phosphorylated antisense strand of AD-59153 (A-119923). Luc (AD-57740) was also tested.

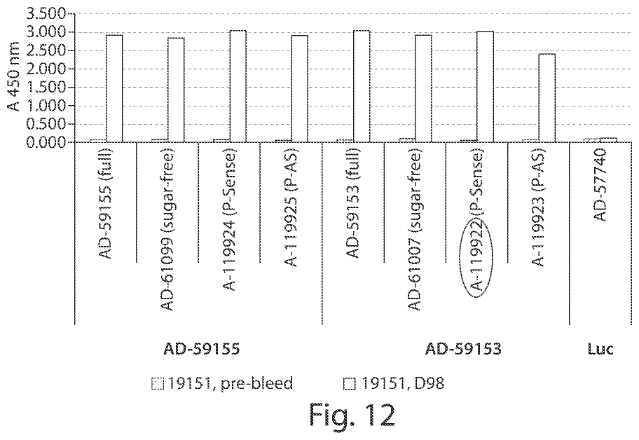

FIG. 12 shows the results when the polyclonal anti-KLH-AD-59153 antibodies from rabbit #19151 (Day 98) were used (diluted 10,000-fold). As shown in FIG. 12, antibodies from this rabbit recognized both AD-59153 and AD-59155 compounds, probably through the 2′fluoro modified nucleotides present in the sequences (circled compound indicates the AD-59153 sense strand that does not contain phosphorothioate). The results also indicated that phosphorothioate containing oligo is not an epitope for the antibody.

These results indicate that animals showed different antibody responses to the same antigen administrated by the same dosing regimen and route.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for detecting an antibody against a nucleic acid molecule in a sample, comprising:
(a) providing a nucleic acid molecule covalently immobilized to a solid support;
(b) contacting said immobilized nucleic acid molecule with the sample under conditions that allow binding of the antibody against the nucleic acid molecule, if present in the sample, to the immobilized nucleic acid molecule, thereby forming a complex of the antibody and the immobilized nucleic acid molecule; and
(c) providing a detection agent that specifically binds to the complex of the antibody and the immobilized nucleic acid molecule under conditions where binding to the complex occurs,
wherein, if the antibody is present in the sample, the binding of the detection agent to the complex of the antibody and the immobilized nucleic acid molecule allows for detection of the antibody in the sample.

2. A method for detecting, or evaluating the level of, an anti-drug antibody (ADA) to a nucleic acid molecule, in a sample, comprising:
(a) providing the sample acquired from a subject;
(b) contacting said sample with a covalently immobilized form of the nucleic acid molecule under conditions that allow binding of the ADA, if present in the sample, to the immobilized form of the nucleic acid molecule, thereby forming a complex of the ADA and the immobilized form of the nucleic acid molecule; and
(c) detecting the complex of the ADA and the immobilized form of the nucleic acid molecule under conditions where binding to the complex is indicative of the presence or level of the ADA, thereby allowing detection or evaluation of the level of ADA in the subject.

3. The method of claim 1, wherein the contacting step is effected using an enzyme-linked immunosorbent assay (ELISA).

4. The method of claim 1, wherein the nucleic acid molecule is chosen from a double stranded RNA (dsRNA) molecule, a single-stranded RNAi molecule, a microRNA (miRNA), an antisense RNA, a short hairpin RNA (shRNA), an iRNA, an mRNA, or a double-stranded oligonucleotide.

5. The method of claim 1, wherein the nucleic acid molecule comprises a sense and an antisense strand.

6. The method of claim 1, wherein the nucleic acid molecule is a dsRNA that forms a duplex structure between 15 and 30 base pairs in length.

7.-9. (canceled)

10. The method of claim 1, wherein the nucleic acid molecule comprises at least one modified nucleotide chosen from one or more of: a 2′-O-methyl modified nucleotide, a nucleotide comprising a 5′-phosphorothioate group, a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, or a non-natural base comprising nucleotide.

11. (canceled)

12. The method of claim 1, wherein at least one end of the nucleic acid molecule comprises a 3′ overhang.

13. The method of claim 12, wherein the 3′ overhang consists of 1 to 5 nucleotides in length.

14. (canceled)

15. The method of claim 1, wherein the nucleic acid molecule has a sequence having an identity of at least 70, 80 or 90 percent or fully complementary to a target mRNA.

16. (canceled)

17. The method of claim 1, wherein the nucleic acid molecule comprises a conjugate of a dsRNA and a ligand.

18. (canceled)

19. The method of claim 17, wherein the ligand comprises one or more N-acetylgalactosamine (GalNAc) ligands, wherein the ligand is attached at the 3′-end, the 5′-end, or both, of the sense and/or the antisense strand of the nucleic acid molecule.

20.-21. (canceled)

22. The method of claim 17, wherein the ligand is attached at the 3′-end of the sense strand of a dsRNA molecule comprising a blunt end.

23. The method of claim 19, wherein the ligand comprises a triantennary GalNAc ligand (GalNAc₃).

24. The method of claim 23, wherein the ligand comprises the following:

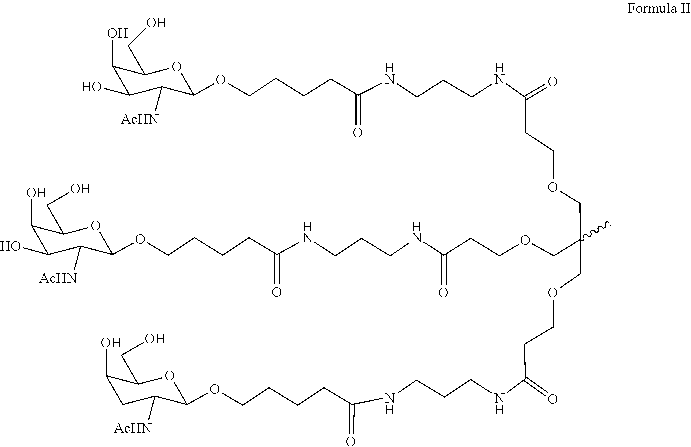 [SMILES: CC(=O)NC1CC(O)C(CO)OC1OCCCCC(=O)NCCCNC(=O)CCOCC(C)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

25. (canceled)

26. The method of claim 1, wherein the target mRNA is chosen from Eg5, PCSK9, TTR, HAMP, VEGF gene, antithrombin 3 gene, aminolevulinate synthase 1 gene, alpha-antitrypsin gene, tmprss6 gene, complement C3 gene, or complement C5 gene.

27. (canceled)

28. The method of claim 1, wherein the solid support is a surface, a plate or a bead.

29. The method of claim 28, wherein the immobilization of the nucleic acid molecule to the solid support provides one or more of stability, a qualitative display of the nucleic acid molecule, a quantitative display of the nucleic acid molecule, a substantially non-denatured nucleic acid molecule, or a nucleic acid molecule conformation that exposes one or more epitopes.

30. The method of claim 1, wherein the sense strand, the antisense strand, or both, is/are covalently coupled to the solid support.

31.-34. (canceled)

35. The method of claim 1, wherein the nucleic acid molecule is phosphorylated at the 5′-end of a sense or an antisense strand, or both.

36. The method of claim 35, wherein the 5′ phosphorylated nucleic acid molecule is immobilized to the solid support via a reactive group chosen from an amine group or a sulfhydryl group.

37. (canceled)

38. The method of claim 36, wherein the phosphate group of the nucleic acid molecule forms a covalent bond with the reactive group present on the solid support or forms a phosphoramidate bond with the secondary amino group present on the solid support.

39. (canceled)

40. The method of claim 1, wherein the nucleic acid molecule is covalently coupled to a polystyrene surface.

41. The method of claim 40, wherein the polystyrene surface is grafted with secondary amino groups at a density between about 10¹⁰/cm² and about 10¹⁶/cm².

42.-43. (canceled)

44. The method of claim 1, wherein the sample comprises plasma, serum, blood, or a non-cellular body fluid.

45.-46. (canceled)

47. The method of claim 1, wherein the sample is acquired prior to, during, or after a treatment with a nucleic acid molecule.

48. The method of claim 1, wherein the detection step comprises a colorimetric means for evaluating the level of the antibody, wherein the colorimetric means is chosen from absorbance, fluorescent intensity or polarization.

49. (canceled)

50. The method of claim 1, wherein the detection step comprises providing a detection agent that specifically binds to the complex of the antibody and the immobilized nucleic acid molecule.

51. The method of claim 50, wherein the detection agent is a detection antibody that binds to the antibody that binds to the nucleic acid molecule if present in the sample.

52.-54. (canceled)

55. The method of claim 50, wherein the detection agent is
(a) a detectably labeled agent chosen from a radiolabeled, a chromophore-labeled, a fluorophore-labeled, or an enzyme-labeled agent, or
(b) an antibody or antibody fragment conjugated to an enzyme or a substrate, or with a protein or ligand of a protein-ligand pair.

56.-62. (canceled)

63. The method of claim 2, wherein the subject has undergone, is undergoing or will receive a therapy that comprises the nucleic acid molecule.

64. A kit for evaluating or detecting an antibody against a nucleic acid molecule, in a sample, comprising:
(a) a nucleic acid molecule covalently immobilized to a solid support;
(b) a detection agent that specifically binds to a complex of the antibody and the immobilized nucleic acid molecule;
(c) instructions for contacting said immobilized nucleic acid molecule with the sample under conditions that allow binding of the antibody, if present in the sample, to the immobilized nucleic acid molecule, and (optionally) instructions for detecting the complex of the antibody and the immobilized nucleic acid molecule.

65.-72. (canceled)

73. A method for evaluating or detecting an antibody against a nucleic acid molecule in a sample, comprising:
(a) providing the nucleic acid molecule;
(b) providing a pre-determined amount of a binding agent that binds to the nucleic acid molecule, wherein the binding agent is radioactively- or fluorescently-labeled;
(c) combining, in solution, the nucleic acid molecule and the binding agent in the presence or the absence of a sample under conditions that allow binding of either the binding agent or the antibody if present in the sample, to the nucleic acid molecule to occur,
thereby evaluating or detecting the antibody against the nucleic acid molecule in solution.

74. The method of claim 73, further comprising determining the amount of a complex between the nucleic acid molecule and the binding agent in the presence or absence of the sample, wherein a decrease in said complex is indicative of the presence or amount of the antibody against the nucleic acid molecule in the sample, and wherein the amount of free binding agent is indicative of the amount of the antibody against the nucleic acid molecule present in the sample.

75. (canceled)

76. The method of claim 73, wherein the combining step is effected in solution using a radioimmunoassay (RIA).

77. The method of claim 73 any of claims 73 76, wherein the binding agent is an antibody molecule that has one or more of the following: binds to the nucleic acid molecule in a sequence-specific manner to a nucleic acid molecule, binds to a fluoro group; or a ligand that includes one or more N-acetylgalactosamine (GalNAc) ligands.


SEQUENCE LISTING (11 sequences)

SEQ ID NO: 1 - protein, 16 aa, Unknown
  source: /note=“Description of Unknown: RFGF peptide”
AAVALLPAVLLALLAP

SEQ ID NO: 2 - protein, 11 aa, Unknown
  source: /note=“Description of Unknown: RFGF analogue peptide”
AALLPVLLAAP

SEQ ID NO: 3 - protein, 13 aa, Human immunodeficiency virus
GRKKRRQRRRPPQ

SEQ ID NO: 4 - protein, 16 aa, Drosophila sp.
RQIKIWFQNRRMKWKK

SEQ ID NO: 5 - skipped

SEQ ID NO: 6 - RNA, 21 nt, Artificial Sequence
  source: /note=“Description of Artificial Sequence: Synthetic oligonucleotide”
ugggauuucauguaaccaaga

SEQ ID NO: 7 - RNA, 23 nt, Artificial Sequence
  source: /note=“Description of Artificial Sequence: Synthetic oligonucleotide”
ucuugguuacaugaaaucccauc

SEQ ID NO: 8 - RNA, 21 nt, Artificial Sequence
  source: /note=“Description of Artificial Sequence: Synthetic oligonucleotide”
gguuaacaccauuuacuucaa

SEQ ID NO: 9 - RNA, 23 nt, Artificial Sequence
  source: /note=“Description of Artificial Sequence: Synthetic oligonucleotide”
uugaaguaaaugguguuaaccag

SEQ ID NO: 10 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
cuuacgcugaguacuucgatt

SEQ ID NO: 11 - DNA, 21 bp, Artificial Sequence
  source: /note=“Description of Artificial Sequence: Synthetic oligonucleotide”
  source: /note=“Description of Combined DNA/RNA Molecule: Synthetic oligonucleotide”
ucgaaguacucagcguaagtt